(19) United States
(12) Patent Application Publication
(10) Pub. No.: US 2026/0347880 A1
(43) Pub. Date: Nov. 5, 2026

(54) KIR 7.1 GENE THERAPY VECTORS AND METHODS OF USING THE SAME

(71) Applicant: Wisconsin Alumni Research Foundation, Madison, WI (US)

(72) Inventors: Bikash Pattnaik, Middleton, WI (US); Pawan Shahi, Madison, WI (US)

(21) Appl. No.: 19/738,180

(22) Filed: Jul. 10, 2026

Related U.S. Application Data
(63) Continuation of application No. 17/284,408, filed on Oct. 10, 2019, now Pat. No. 12,709,760.
(60) Provisional application No. 62/743,623, filed on Oct. 10, 2018.

(51) Int. Cl. C12N 15/86; A61K 9/00; A61K 48/00; C07K 14/705

(52) U.S. Cl. CPC C12N 15/86; A61K 9/0048; C07K 14/705; A61K 48/00; C12N 2740/15043; C12N 2740/15071; C12N 2750/14143; C12N 2750/14171

(57) ABSTRACT

The present invention is directed to gene therapy constructs and pharmaceutical compositions for the expression of Kir7.1. The gene therapy constructs include a vector comprising a promoter operably connected to a polynucleotide encoding a Kir7.1 polypeptide. Methods of treating a subject having a condition associated with insufficient expression or function of a Kir7.1 polypeptide are also provided.

### CROSS-REFERENCE TO RELATED APPLICATIONS

This application is a continuation of U.S. application Ser. No. 17/284,408, filed Apr. 9, 2021, which is a national stage filing under 35 U.S.C. § 371 of International Application No. PCT/US2019/055635, filed Oct. 10, 2019, which claims benefit to U.S. Provisional Application No. 62/743,623 filed on Oct. 10, 2018. The contents of these applications are incorporated by reference herein.

### STATEMENT REGARDING FEDERALLY SPONSORED RESEARCH

This invention was made with government support under EY024995 awarded by the National Institutes of Health. The government has certain rights in the invention.

### SEQUENCE LISTING

A Sequence Listing accompanies this application and is submitted as an XML file of the sequence listing named “96029604752_SL.xml” which is 50,349 bytes in size and was created on Jul. 10, 2026. The sequence listing is electronically submitted via Patent Center with the application and is incorporated herein by reference in its entirety.

### INTRODUCTION

Leber congenital amaurosis (LCA) is an inherited pediatric form of blindness characterized by severe loss of vision at birth. Children with LCA may also exhibit a variety of other abnormalities including roving eye movements (nystagmus), deep-set eyes, sensitivity to bright light, and central nervous system abnormalities. Typically, within an infant's first few months of life, parents notice a lack of visual responsiveness and nystagmus. Although the retinas of infants with LCA appear normal, little (if any) activity is detected in the retina by electroretinography (ERG). By early adolescence, however, various changes in the appearance of retina may be detected including pigmentary changes in the retinal pigment epithelium (RPE) and the presence of constricted blood vessels.

LCA is typically passed through families in an autosomal recessive pattern of inheritance. Mutations in at least 21 genes that are expressed in the outer retinal photoreceptors and retinal pigment epithelium (RPE) have been associated with LCA. Within the last decade, autosomal recessive mutations in the human KCNJ13 gene (603203 on chromosome locus 2q37.1) have been identified in patients with a specific form of LCA known as LCA16. To date, LCA16 pathogenic allelic variants include c. 158G>A (p.Trp53Ter), c.359T>C (p.Iso120Thr), c.458C>T (p.Thr153Iso), c.496C>T (p.Arg166Ter), and c.722T>C (p.Leu241Pro). In addition, the compound heterozygous KCNJ13 mutations c.314 G>T (p.Ser105Iso) and c.655C>T (p.G219Ter) are known to cause early-onset retinal dystrophy in an LCA patient⁵. An autosomal dominant KCNJ13 mutation, c.484C>T (p.Arg162Trp), causes early-onset blindness called snowflake vitreoretinal degeneration (SVD OMIM-193230).

The human KCNJ13 gene encodes an inward rectifying potassium channel—Kir7.1. The Kir7.1 protein is expressed in several human tissues including the cell apical processes of RPE, in which it modulates retinal function and health. The role of the Kir7.1 channel in other organs remains to be elucidated.

Although the role of Kir7.1 is beginning to be understood in conditions such as LCA16, there are no approved therapies to treat channelopathies or conditions associated with insufficient expression or function of the Kir7.1 protein. Accordingly, there is a need in the art for new therapies for treating such conditions.

### SUMMARY

In one aspect of the present invention, gene therapy vectors are provided. The gene therapy vectors may include a promoter operably connected to a polynucleotide encoding a Kir7.1 polypeptide.

In another aspect, the present invention relates to therapeutic compositions. The therapeutic compositions may include any of the gene therapy vectors described herein and a pharmaceutically-acceptable carrier.

In a further aspect of the present invention, methods of treating a subject having a condition associated with insufficient expression or function of a Kir7.1 polypeptide are provided. The methods may include administering a therapeutically effective amount of any one of the gene therapy vectors described herein or any one of the therapeutic compositions described herein to the subject.

### BRIEF DESCRIPTION OF THE DRAWINGS

The patent or patent application file contains at least one drawing in color. Copies of this patent or patent application publication with color drawings will be provided by the Office upon request and payment of the necessary fee.

FIGS. 1A-1N show patient-derived iPSC-RPE with the LCA16 phenotype. (FIG. 1A) Illustration of a mature RPE cell (bright-field image) with the normal TGG sequence (SEQ ID NO:19). Family pedigree indicating sample origin. (FIG. 1B) Bright-field image of mature RPE cells derived from an LCA16 proband with the TAG sequence. (SEQ ID NO:20) (FIG. 1C) Normal karyotype in the patient sample with no clonal abnormalities. (FIG. 1D) Analysis of the Nhe1 digestion product from the control, LCA16, and wild-type iPSC-RPE lines and human fetal RPE cells. The full-length Kir7.1 sequence is 1083 bp in length, and the digested products are 925 and 158 bp in length. (FIG. 1E) RPE cell-specific gene expression in iPSC-RPE cells. (FIG. 1F) Electron micrograph of a representative LCA16 iPSC-RPE cell. (FIG. 1G) Comparison of the average mitochondria (Mit) count within 10 μm of the cell. (FIG. 1H) Evaluation of the average length of RPE apical (AP) processes. (FIG. 1I) Immunofluorescence localization of Kir7.1 (red), ZO-1 (green) and DAPI (blue) in control iPSC-RPE cells. Both the lower and side panels reveal a polarized distribution of Kir7.1 in reference to ZO-1 and DAPI (z-stack images). (FIG. 1J) Localization of Kir7.1 (red), ZO-1 (green) and DAPI (blue) in LCA16 iPSC-RPE. (FIG. 1K) Western blot results showing the expression of RPE cell-specific proteins in both tissue samples. Using a C terminal-specific antibody against Kir7.1, we detected Kir7.1 protein in whole-cell lysates from the control iPSC-RPE but not in those from the LCA16 iPSC-RPE. Phagosomes (red) localization within control iPSC-RPE (FIG. 1L) and LCA16 iPSC-RPE (FIG. 1M) samples. (FIG. 1N) Plot of the average phagosome count within a fixed 200 μm² area in the control and diseased iPSC-RPE cells after 4 hr of feeding and a subsequent 48-hr digestion period or after 1 day of feeding followed by 6 days of digestion.

FIGS. 2A-2N show a putative Kir7.1 loss-of-function cure through nonsense mutation suppression or gene augmentation. (FIG. 2A) Plot of the average current-voltage (I/V) curve for Kir7.1 currents using normal external K+ (black) or high external Rb+ (light blue) in control iPSC-RPE cells. (FIG. 2B) An average I/V curve using normal K+ (red) and high Rb+ (light blue) in LCA16 iPSC-RPE cells. (FIG. 2C) Average plot of an inward current amplitude measured at −150 mV. Color representation as shown in a and b. (FIG. 2D) Comparison of the average membrane potential of the control (black) cells to depolarized LCA16 (red) RPE cells. (FIG. 2E) Average I/V relationship before (red) and after (dark blue) treatment with NB84. The current measured in Rb+ is shown as a light-blue trace. Evaluation of the average inward current measured at −150 mV (FIG. 2F) and membrane potential (FIG. 2G) to demonstrate the effect of NB84. (FIG. 2H) A GFP-fused protein was precipitated using anti-GFP antibody as a trap, and silver staining shows the purified component bands for the full-length Kir7.1 and W53X proteins. The GFP control sample shows a smaller protein product. (FIG. 2I) Western blot analysis of cell lysates shows the respective bands when probed with a GFP-specific antibody. A partial restoration of the full-length protein product is observed after NB84 treatment. (FIG. 2J) Plot of the average I/V curve for Kir7.1 currents measured in GFP-positive cells expressing a normal copy of the human Kir7.1 clone. Both K+ (green) and Rb+ (light blue) traces are shown. Average plot of the current amplitudes (FIG. 2K) measured at −150 mV and membrane potential (FIG. 2L) to show rescue after gene augmentation. (FIG. 2M) Cultured LCA16 iPSC-RPE showing wild-type Kir7.1 (green), ZO-1 (red) and DAPI (blue) proteins. Z-stack planes are shown in the lower and side panels. (FIG. 2N) Western blot analysis of Kir7.1 protein expression in LCA16 iPSC-RPE cells detected after gene augmentation by using anti-GFP antibody.

FIGS. 3A-3D show the phenotype of patient-derived iPSC-RPE cells. Comparison of electron micrograph of a control hiPSC-RPE cell (FIG. 3A) and an LCA16 hiPSC-RPE cell (FIG. 3B) showing normal columnar morphology with basal infoldings, large nuclei, mitochondria (m), melanosomes and intact apical membrane with extending processes (ap). Images of live control hiPSC-RPE (FIG. 3C) and patient-derived hiPSC-RPE (FIG. 3D) cells in x-y-z dimension showing POS (red) and nuclei (blue) imaged 6 days after feeding cells for 1 day with fluorescent-labeled bovine POS. More undigested red fluorescent POS particles are visible in LCA16 hiPSC-RPE cells.

FIGS. 4A-4D show that a subpopulation of hiPSC-RPE show rescue in membrane potential but not current amplitude. (FIG. 4A) I/V plot of average current response in a subgroup of LCA16 hiPSC-RPE cells in normal K+ Ringer's and high Rb+ Ringer's solution after treatment of cells with 100 μM NB84. (FIG. 4B) I/V plot showing K+ and Rb+ current response in LCA16 hiPSC-RPE after treatment with 500 μM NB84. (FIG. 4C) Average plot of membrane potential showing rescue of membrane potential to control levels after treatment of LCA16 hiPSC-RPE with 100 or 500 μM NB84. (FIG. 4D) Current amplitude plot clearly demonstrating no rescue in current amplitude after treatment with either 100 or 500 μM NB84.

FIGS. 5A-5D show read-through of Trp53Ter ectopically expressed in CHO cells. As in LCA16 hiPSC-RPE cells, transduced CHO cells showed inwardly rectifying Kir7.1 current activated by Rb+ only after treatment with NB84. (FIG. 5A) I/V plot of cells showing K+ (black) and Rb+ (red) current after treatment with NB84 showing recovery of both current amplitude and membrane potential. (FIG. 5B) A group of NB84 treated cells showing somewhat linear I/V plot for K+ (black) and Rb+ (red) illustrating recovery of only membrane potential but not current amplitude. Comparison of average recovery of both current amplitude (FIG. 5C) and membrane potential (FIG. 5D) after treatment of Trp53Ter expressing CHO cells.

FIGS. 6A-6D show determination of the extent of wildtype protein expression required for functional rescue. We were particularly interested in quantitating how much gene augmentation/correction is required to restore channel function. We expressed either Trp53Ter or wild type Kir7.1 protein alone or in various combinations in CHO cells. (FIG. 6A) Current recordings are shown as I/V plots. (FIG. 6B) On an expanded scale for x-axis, resting membrane potential shows negative shift with wild type protein making up only 20% of the protein expression. (FIG. 6C) Average plot of either normalized current amplitude (filled circles) or membrane potential (grey bar) as a function of increasing wildtype protein expression. Solid line is a best fit for distribution using equation shown in FIG. 6D. Half-maximum current was obtained with about 26% of the wild type protein expression. (FIG. 6D) Values of best fit curve indicating half maximal response and Hill Slope.

FIGS. 7A-7D show a comparison of the rescue of membrane potential across treatment modalities. (FIG. 7A) On an expanded scale of the x-axis, resting membrane potential of control (black) and LCA16 iPSC-RPE (red) showed a positive shift in I/V plot. (FIG. 7B) For the LCA16 iPSC-RPE cells (red trace), treatment with NB84 shifted the I/V-plot to negative (blue). (FIG. 7C) Plot of average I/V also showed a negative shift of resting potential after gene augmentation (green). (FIG. 7D) Bar graph comparison of resting membrane potential showed recovery of LCA16 iPSC-RPE to control level after treatment with NB84 or upon gene augmentation.

FIG. 8 shows whole-cell current voltage relationship from wildtype (left panel) and W53X mutant (right panel) stable cells. Inwardly rectifying K+ current (black trace) in the wildtype stable cell was significantly increased by Rb+ (red trace). In the W53X mutant stable cells on the right, neither K+ nor Rb+ current was recorded (p=1.05E−0.5).

FIG. 9A-9E shows gene augmentation of W53X mutant expressing CHO cells had recovery of average inwardly rectifying K+ current (FIG. 9A. IV plot in red trace) compared to no current before (FIG. 9A. plot in black trace). (FIG. 9B) Average higher Rb+ current (red trace) in W53X mutant expressing cells after gene augmentation. (FIG. 9C) Net increase in Rb+ permeability increased (Blue) through Kir7.1 channel after gene augmentation. (FIG. 9D) Complete recovery of resting membrane potential (RMP) after AAV-Kir7.1 transduction of W53X expressing cells represented as blue box. (FIG. 9E) Western blot results showing expression of full length protein product after gene augmentation in lane W53X+AAV (red band).

FIG. 10A-10B shows Kir7.1 expression (green) in W53X mutant line after gene augmentation through AAV-Kir7.1 (FIG. 10A). (FIG. 10B) A higher magnification image shows membrane localization of the Kir7.1 protein alongside membrane marker WGA-Alexa 594. In the lower panel is the line scan for red and green showing membrane marker and Kir7.1 co-localize.

FIG. 11 shows Kir7.1 gene-therapy in vivo. On the left is a control mouse showing normal wave form of electroretinogram and no change after gene augmentation. In the middle is a conditional knock out mice showing no c-wave in the right black trace. This wave which directly depends on Kir7.1 expression is completely recovered 4 weeks after gene therapy. Average result is shown in box plot with significant recovery of c-wave in experimental gene therapy.

FIG. 12 shows a vector map for an exemplary AAV viral vector for delivery of a Kir7.1 protein.

FIG. 13 shows a vector map for an exemplary Lentivirus viral vector for delivery of a Kir7.1 protein.

FIGS. 14A-14F demonstrates functional recovery of Retinal Pigment Epithelial (RPE) cells lacking Kir7.1 protein after gene therapy. (FIG. 14A) Injection control on WT mice and the cKO control mice depicting the RPE response functional after 8 weeks with PBS injection. (FIG. 14B) ERG response from the Kir7.1 cKO mice which showed no a-, b- and c-wave during the screening. Delivery of the Kir7.1 with lentivirus carrying either constitutive EF1a promoter or RPE specific VMD2 promoter failed to rescue the RPE function due to the severe phenotype as both RPE and photoreceptors were degenerated. (FIG. 14C) c-wave from RPE is recovered in the cKO mice, by subretinal delivery of lentivirus carrying kcnj13 gene driven by EF1a and VMD2 promoter, where the photoreceptors were not degenerated but had no response from the RPE cells during screening. (FIG. 14D), (FIG. 14E), (FIG. 14F) Representative optical coherence tomography (OCT) images showing the retinal structure from the control mice, cKO mice (no-a-,b-c-wave) with no recovery and c-wave recovered mice (a-, b- but no-c-wave) during screening and post 8 weeks after lentiviral gene delivery respectively.

FIGS. 15A-15E demonstrates results of a subset of mice that did not show c-wave recovery. (FIG. 15A) Graph representing the subset of mice that did not show c-wave recovery after injection of lentivirus carrying kcnj13 gene driven by EF1a and VMD2 promoter. (FIG. 15B), (FIG. 15C) Optical coherence tomography (OCT) image of cKO mice with no c-wave during screening shows intact retina but wanes after 8 weeks revealing the progressive nature of retina degeneration over time due to the lack of Kir7.1 protein in RPE cell. (FIG. 15D), (FIG. 15E) OCT images showing the retinal structure from cKO mice those having the response from photoreceptors (a- and b-wave) but lacking c-wave response from RPE. Injection of the lentivirus carrying the kcnj13 gene failed to restore c-wave, could be due to inefficiency of the RPE transduction or mutilation due to injection.

FIG. 16 depicts Table 2 and Table 3 demonstrating an exemplary AAV vector for the present invention containing specific components and a suitable exemplary sequence for the AAV vector comprising a RPE specific promoter and the Kir7.1 gene.

FIG. 17 depicts Table 5 and Table 6 demonstrating an exemplary lentiviral vector of the present invention, including the specific components and a suitable exemplary sequence for the lentiviral vector.

### DETAILED DESCRIPTION

Here, the present inventors disclose new gene therapy vectors and therapeutic compositions that may be used to treat Leber Congenital Amaurosis 16 (LCA16) or other conditions associated with insufficient expression or function of a Kir7.1 protein. In the non-limiting Examples, the inventors surprisingly show that a gene therapy approach may be used to effectively restore Kir7.1 polypeptide function in retinal pigment epithelium (RPE) cells either in vitro or in vivo resulting in RPE cells with rescued electrophysiological phenotypes. The inventors thus have discovered that gene therapy approaches may be used to effectively deliver the membrane protein Kir7.1. The present inventors demonstrate in part that expression of a Kir7.1 protein open reading frame alone is sufficient to get the Kir7.1 protein trafficked to the proper subcellular compartment. These results provide hope for potential curative therapeutics to treat Leber Congenital Amaurosis 16 (LCA16) or other conditions associated with insufficient expression or function of a Kir7.1 protein.

### Gene Therapy Vectors

In one aspect of the present invention, gene therapy vectors are provided. The gene therapy vectors may include a promoter operably connected to a polynucleotide encoding a Kir7.1 polypeptide. The general approach in certain aspects of the present invention is to provide a cell with an expression construct encoding a Kir7.1 polypeptide, thereby permitting the expression of the Kir7.1 polypeptide in the cell. Following delivery of the expression construct, the Kir7.1 polypeptide encoded by the expression construct is synthesized by the transcriptional and translational machinery of the cell.

As used herein, an “expression construct encoding a Kir7.1 polypeptide” refers to a promoter operably connected to a polynucleotide encoding a Kir7.1 polypeptide.

As used herein, the terms “polynucleotide,” “polynucleotide sequence,” “nucleic acid” and “nucleic acid sequence” refer to a nucleotide, oligonucleotide, polynucleotide (which terms may be used interchangeably), or any fragment thereof. These phrases also refer to DNA or RNA of natural or synthetic origin (which may be single-stranded or double-stranded and may represent the sense or the antisense strand). In some embodiments, the promoters and Kir7.1 polynucleotides or expression constructs encoding a Kir7.1 polypeptide described herein are encoded in double-stranded DNA, single-stranded DNA, or RNA.

As used herein, a “gene therapy vector” refers to viral or non-viral vector systems that may be used to deliver an expression construct encoding a Kir7.1 polypeptide into a cell (i.e., eukaryotic cell). Both broad types of vector systems are described in the following sections. There also are two primary approaches utilized in the delivery of an expression construct for the purposes of gene therapy; either indirect, ex vivo methods or direct, in vivo methods. Ex vivo gene transfer comprises vector modification of (host) cells in culture and the administration or transplantation of the vector modified cells to a gene therapy recipient. In vivo gene transfer comprises direct introduction of the vector (e.g., injection, inhalation) into the target source or therapeutic gene recipient.

In certain embodiments of the invention, the expression construct encoding the Kir7.1 polypeptide may be stably integrated into the genome of the cell. In yet further embodiments, the expression construct encoding the Kir7.1 polypeptide may be stably or transiently maintained in the cell as a separate, episomal segment of DNA. Such nucleic acid segments or “episomes” encode sequences sufficient to permit maintenance and replication independent of or in synchronization with the host cell cycle. How the expression construct is delivered to a cell and/or where in the cell the nucleic acid remains is dependent on the type of vector employed. The following gene delivery methods provide the framework for choosing and developing the most appropriate gene delivery system for a preferred application.

### Non-Viral Gene Therapy Vectors

In some embodiments, the gene therapy vector may be a delivery particle. Delivery particles suitable for delivering polynucleotides are known in the art and may include, without limitation, polymeric particles, liposomal particles, and particles including lipids and at least one type of polymer. In some embodiments, the delivery particles may be formed using common Lipofectamine reagents.

The delivery particles may include nanoscale particles and/or microscale particles, for example, as delivery vehicles of components to a cell for genome editing. The particles may have an effective average diameter less than about 500 μm, 100 μm, 50 μm, 20 μm, 10 μm, 5 μm, 2 μm, 1 μm, 0.5 μm, 0.2 μm, 0.1 μm, 0.05 μm, 0.02 μm, 0.01 μm, or have an effective average diameter within a range bounded by any of 500 μm, 100 μm, 50 μm, 20 μm, 10 μm, 5 μm, 2 μm, 1 μm, 0.5 μm, 0.2 μm, 0.1 μm, 0.05 μm, 0.02 μm, 0.01 μm (e.g., 0.01-5 μm). The nanoscale particles and microscale particles may be referred to as “nanoparticles” and “microparticles,” respectively.

Polymeric particles have been described in the art. (U.S. Patent Publication 20140066388). Polymeric particles may include or may be formed from biodegradable polymeric molecules, which in some embodiments may include dendrimers. Suitable dendrimers may include, but are not limited to, polyamidoamine (PAMAM) dendrimers. Polyamidoamine dendrimers have been used in the art as vehicles for intracellular delivery of therapeutics. Polyamidoamine dendrimers suitable for preparing the presently disclosed nanoparticles may include 3rd-, 4th-, 5th-, or preferably at least 6th-generation dendrimers.

Polymeric particles may also include or may be formed from other biodegradable polymeric molecules which may include, without limitation, polylactic acid (PLA), polygycolic acid (PGA), co-polymers of PLA and PGA (e.g., polyactic-co-glycolic acid (PLGA)), poly-ε-caprolactone (PCL), polyethylene glycol (PEG), poly(3-hydroxybutyrate), poly(p-dioxanone), polypropylene fumarate, poly(orthoesters), polyol/diketene acetals addition polymers, poly-alkyl-cyano-acrylates (PAC), poly(sebacic anhydride) (PSA), poly(carboxybiscarboxyphenoxyphenoxy hexone (PCPP) poly[bis(p-carboxypheonoxy) methane] (PCPM), copolymers of PSA, PCPP and PCPM, poly(amino acids), poly(pseudo amino acids), polyphosphazenes, derivatives of poly[(dichloro)phosphazenes] and poly[(organo)phosphazenes], poly-hydroxybutyric acid, or S-caproic acid, elastin, gelatin, and chitosan. (See, e.g., Kumari et al., Colloids and Surfaces B: Biointerfaces 75 (2010) 1-18; and U.S. Pat. Nos. 6,913,767; 6,884,435; 6,565,777; 6,534,092; 6,528,087; 6,379,704; 6,309,569; 6,264,987; 6,210,707; 6,090,925; 6,022,564; 5,981,719; 5,871,747; 5,723,269; 5,603,960; and 5,578,709; and U.S. Published Application No. 2007/0081972; and International Application Publication Nos. WO 2012/115806; and WO 2012/054425). In some embodiments, the particles may include a mixture of PLGA and PAMAM.

Polymeric particles may be prepared by methods known in the art. (International Application Publication Nos. WO 2012/115806; and WO 2012/054425). Suitable methods for preparing the nanoparticles may include methods that utilize a dispersion of a preformed polymer, which may include but are not limited to solvent evaporation, nanoprecipitation, emulsification/solvent diffusion, salting out, dialysis, and supercritical fluid technology. In some embodiments, the nanoparticles may be prepared by forming a double emulsion (e.g., water-in-oil-in-water) and subsequently performing solvent-evaporation. The nanoparticles obtained by the disclosed methods may be subjected to further processing steps such as washing and lyophilization, as desired. Optionally, the nanoparticles may be combined with a preservative (e.g., trehalose).

Micelle and liposomal-based particles may also serve as suitable delivery particles. See, e.g., U.S. Pat. No. 8,252,324. Micelles are self-assembling spherical colloidal nanoparticles formed by amphiphilic molecules. Micelles are also described as aggregate surfactant molecules disbursed in a liquid colloid. The core of the micelle, which is segregated in an aqueous milieu, is capable of encapsulating polynucleotides and/or proteins protecting them from destruction and biological surroundings while improving their pharmacokinetics and biodistribution. Micelles are generally in the order of 5-50 nm in diameter, and are therefore capable of accumulating in pathological areas with leaky vasculature, such as infarct zones and tumors due to the enhanced permeability and retention effect. Micelles are also capable of evading a major obstacle in drug targeting by particulate systems: non-specific uptake by the reticulo-endothelial systems and renal secretion. In contrast to micelles, liposomes are bilayered phospholipid vesicles approximately 50 to 1,000 nm in diameter. Liposomes are biologically inert and completely biocompatible; they cause practically no toxic or antigenic reactions. Polynucleotides included in liposomes are protected from the destructive action of the external media by the liposomes. Thus, liposomes are able to deliver their content inside cells and even inside different cell compartments. Generally, liposomes are considered a promising carrier with significant therapeutic potential, as demonstrated in numerous laboratory tests and clinical trials.

Delivery particles may also include particles including lipids and polymer components. For example, particles including a phospholipid bilayer and poly(beta-amino ester) (PBAE) have been developed for the in vivo delivery of polynucleotides.

The delivery particles preferably have physical properties that facilitate uptake by a targeted cell. For example, preferably the particles have a size and a charge that facilitate uptake by a targeted cell. Typically, the particles have a mean effective diameter of less than 1 micron, and preferably the particles have a mean effective diameter of between about 25 nm and about 500 nm, and more preferably between about 50 nm and about 250 nm, and most preferably about 100 nm to about 150 nm. The size of the particles (e.g., mean effective diameter) may be assessed by known methods in the art, which may include but are not limited to transmission electron microscopy (TEM), scanning electron microscopy (SEM), Atomic Force Microscopy (AFM), Photon Correlation Spectroscopy (PCS), Nanoparticle Surface Area Monitor (NSAM), Condensation Particle Counter (CPC), Differential Mobility Analyzer (DMA), Scanning Mobility Particle Sizer (SMPS), Nanoparticle Tracking Analysis (NTA), X-Ray Diffraction (XRD), Aerosol Time of Flight Mass Spectroscopy (ATFMS), and Aerosol Particle Mass Analyzer (APM).

Delivery particles will be taken up by cells non-specifically even if the particles do not include a specific ligand on their surface. However, the disclosed delivery particles may be configured to also include a ligand that specifically targets a particular cell type. In order to achieve more specific targeting of delivery particles, such particles may be modified with various ligands using advanced conjugation procedures. For example, antibodies and small peptides have been attached to the water exposed tips of polyethyleneglycol chains. Antibodies and small peptides have also been conjugated via reactive p-nitrophenylcarbonyl, N-benzotrazole carbonyl or maleimide terminated PEG-phosphatidylethanolamine.

### Viral Gene Therapy Vectors

The gene therapy vector may also be a viral vector. The viral vector may be a virus particle or may be encoded on a DNA plasmid. In some embodiments where the viral vector is a virus particle, for example a lentivirus viral particle, the virus particle may include a VSV-G envelop protein. The capacity of certain viral vectors to efficiently infect or enter cells, to integrate into a host cell genome and stably express viral genes, have led to the development and application of a number of different viral vector systems (Robbins et al., 1998). Viral systems are currently being developed for use as vectors for ex vivo and in vivo gene transfer. For example, adenovirus, herpes-simplex virus, retrovirus and adeno-associated virus vectors are being evaluated currently for treatment of human diseases. The various viral vectors described below present specific advantages and disadvantages depending on the particular gene-therapeutic application.

Suitable viral vectors that may be used in accordance with the present invention may include, without limitation, retroviral vectors, adeno-associated viral (AAV) vectors, adenoviral vectors, or herpes-simplex vectors. Retroviral vectors may include, for example, lentiviral vectors.

Here, in the non-limiting Examples, the present inventors demonstrate that a polynucleotide encoding a Kir7.1 polypeptide could successfully be introduced and expressed in retinal pigment epithelium (RPE) cells either in vitro or in vivo using either lentiviral or adeno-associated viral (AAV) vectors so as to rescue functional defects in a KCNJ13 gene. Accordingly, in some embodiments, the viral vector may be a lentiviral vector or an AAV, suitably an AAV2, vector. The AAV vectors described herein may further include at least one, two, three, four, five, six, seven, or eight of the components listed in Table 2 or Table 3. The lentiviral vectors described herein may further include at least one, two, three, four, five, six, seven, eight, nine, ten, eleven, twelve, thirteen, fourteen, or fifteen of the components listed in Table 5 or Table 6.

### Retroviral Vectors

In certain embodiments of the invention, the use of retroviruses for gene delivery of the Kir7.1 expression construct is contemplated. Retroviruses or retroviral vectors are RNA viruses comprising an RNA genome. When a host cell is infected by a retrovirus, the genomic RNA is reverse transcribed into a DNA intermediate which is integrated into the chromosomal DNA of infected cells. This integrated DNA intermediate is referred to as a provirus. A particular advantage of retroviruses is that they can stably infect dividing cells with a gene of interest (e.g., a therapeutic gene) by integrating into the host DNA, without expressing immunogenic viral proteins. Theoretically, the integrated retroviral vector will be maintained for the life of the infected host cell, expressing the gene of interest.

Lentiviral vectors are a type of retrovirus that can infect both dividing and nondividing cells. Lentiviruses can be used to provide highly effective gene therapy as lentiviruses can change the expression of their target cell's gene for up to six months. They can be used for nondividing or terminally differentiated cells such as neurons, macrophages, hematopoietic stem cells, retinal photoreceptors, and muscle and liver cells, cell types for which previous gene therapy methods could not be used.

### Adeno-Associated Viral (AAV) Vectors

Adeno-associated virus (AAV), a member of the parvovirus family, is a human virus that is increasingly being used for gene delivery therapeutics. AAV has several advantageous features not found in other viral systems. First, AAV can infect a wide range of host cells, including non-dividing cells. Second, AAV can infect cells from different species. Third, AAV has not been associated with any human or animal disease and does not appear to alter the biological properties of the host cell upon integration. For example, it is estimated that 80-85% of the human population has been exposed to AAV. Finally, AAV is stable at a wide range of physical and chemical conditions which lends itself to production, storage and transportation requirements.

The AAV genome is a linear, single-stranded DNA molecule containing 4681 nucleotides. The AAV genome generally comprises an internal non-repeating genome flanked on each end by inverted terminal repeats (ITRs) of approximately 145 bp in length. The ITRs have multiple functions, including origins of DNA replication, and as packaging signals for the viral genome. AAV ITRs may be derived from any of several AAV serotypes, including AAV1, AAV2, AAV3, AAV4, AAV5, AAV6, AAV7, AAV8, AAV9, AAV10, AAV11, avian AAVs, bovine AAVs etc. The 5′ and 3′ ITRs of the AAV viral vectors disclosed herein may be derived from any of these AAV serotypes. The 5′ and 3′ ITRs which flank the AAV viral vectors disclosed herein need not necessarily be identical or derived from the same AAV serotype. Thus, rAAV vector design and production allow for exchanging the capsid proteins between different AAV serotypes. Homologous vectors comprising an expression cassette flanked by e.g., AAV2-ITRs and packaged in an AAV2 capsid, can be produced as well as heterologous, hybrid vectors where the transgene expression cassette is flanked by e.g., AAV2 ITRs, but the capsid originates from another AAV serotype such as AAV5 for example. Suitably, in some embodiments, the present inventors have found that AAV2 viral vectors may be used to effectively deliver Kir7.1 expression constructs into cells.

The internal non-repeated portion of the AAVgenome includes two large open reading frames, known as the AAV replication (rep) and capsid (cap) genes. The rep and cap genes code for viral proteins that allow the virus to replicate and package the viral genome into a virion. A family of at least four viral proteins is expressed from the AAV rep region, Rep 78, Rep 68, Rep 52, and Rep 40, named according to their apparent molecular weight. The AAV cap region encodes at least three proteins, VP1, VP2, and VP3.

AAV is a helper-dependent virus requiring co-infection with a helper virus (e.g., adenovirus, herpesvirus or vaccinia) in order to form AAV virions. In the absence of co-infection with a helper virus, AAV establishes a latent state in which the viral genome inserts into a host cell chromosome, but infectious virions are not produced. Subsequent infection by a helper virus “rescues” the integrated genome, allowing it to replicate and package its genome into infectious AAV virions. Although AAV can infect cells from different species, the helper virus must be of the same species as the host cell (e.g., human AAV will replicate in canine cells co-infected with a canine adenovirus).

AAV has been engineered to deliver genes of interest by deleting the internal non-repeating portion of the AAV genome and inserting a heterologous gene between the ITRs. The heterologous gene may be functionally linked to a heterologous promoter (constitutive, cell-specific, or inducible) capable of driving gene expression in target cells. To produce infectious recombinant AAV (rAAV) containing a heterologous gene, a suitable producer cell line is transfected with a rAAV vector containing a heterologous gene. The producer cell is concurrently transfected with a second plasmid harboring the AAV rep and cap genes under the control of their respective endogenous promoters or heterologous promoters. Finally, the producer cell is infected with a helper virus. Once these factors come together, the heterologous gene is replicated and packaged as though it were a wild-type AAV genome. When target cells are infected with the resulting rAAV virions, the heterologous gene enters and is expressed in the target cells. Because the target cells lack the rep and cap genes and the adenovirus helper genes, the rAAV cannot further replicate, package or form wild-type AAV.

Suitable AAV vectors are known in the art. For example, suitable AAV vectors include AAV2/5, demonstrated in “AAV2/5-mediated gene therapy in iPSC-derived retinal pigment epithelium of a choroideremia patient”, incorporated by reference in its entirety. See, e.g., Cereso et. al. Mol Ther Methods Clin Dev. 2014. Further examples of AAV vectors that can suitably be adapted for the present gene delivery can be found in “Comparative AAV-eGFP Transgene Expression Using Vector Serotypes 1-9, 7m8, and 8b in Human Pluripotent Stem Cells, RPEs, and Human and Rat Cortical Neurons.” See Duong et. al. Stem Cells Int. 2019.

### Adenoviral Vectors

In particular embodiments, an adenoviral vector is contemplated for the delivery of Kir7.1 expression constructs. “Adenoviral vector” is meant to include those constructs containing adenovirus sequences sufficient to (a) support packaging of the construct and (b) to ultimately express construct that has been cloned therein.

Adenoviruses comprise linear double stranded DNA, with a genome ranging from 30 to 35 kb in size. An adenoviral vector according to the present invention comprises a genetically engineered form of the adenovirus. Advantages of adenoviral gene transfer include the ability to infect a wide variety of cell types, including non-dividing cells, a mid-sized genome, ease of manipulation, high infectivity and they can be grown to high titers. Further, adenoviral infection of host cells does not result in chromosomal integration because adenoviral DNA can replicate in an episomal manner, without potential genotoxicity associated with other viral vectors. Adenoviruses also are structurally stable and no genome rearrangement has been detected after extensive amplification. An exemplary adenoviral vector according to the present invention is replication defective vector that will not have an adenovirus E1 region. Adenovirus growth and manipulation is known to those of skill in the art, and exhibits broad host range in vitro and in vivo. See, e.g., U.S. Pat. Nos. 5,670,488; 5,932,210; 5,824,544.

### Herpes-Simplex Viral Vectors

Herpes simplex virus (HSV) type I and type II contain a double-stranded, linear DNA genome of approximately 150 kb, encoding 70-80 genes. Wild type HSV are able to infect cells lytically and to establish latency in certain cell types (e.g., neurons). Similar to adenovirus, HSV also can infect a variety of cell types. For use in therapeutic gene delivery, HSV must be rendered replication-defective. Protocols for generating replication-defective HSV helper virus-free cell lines have been described (U.S. Pat. Nos. 5,879,934; 5,851,826, each specifically incorporated herein by reference in its entirety).

### Other Viral Vectors

The development and utility of viral vectors for gene delivery is constantly improving and evolving. Other viral vectors such as poxvirus; e.g., vaccinia virus, alpha virus; e.g., sindbis virus, Semliki forest virus, reovirus and influenza A virus are contemplated for use in the present invention and may be selected according to the requisite properties of the target system.

### Promoters

As used herein, the terms “promoter,” “promoter region,” or “promoter sequence” refer generally to transcriptional regulatory regions of a gene, which may be found at the 5′ or 3′ side of the polynucleotides described herein, or within the coding region of the polynucleotides, or within introns in the polynucleotides. Typically, a promoter is a DNA regulatory region capable of binding RNA polymerase in a cell and initiating transcription of a downstream (3′ direction) coding sequence. The typical 5′ promoter sequence is bounded at its 3′ terminus by the transcription initiation site and extends upstream (5′ direction) to include the minimum number of bases or elements necessary to initiate transcription at levels detectable above background. Within the promoter sequence is a transcription initiation site (conveniently defined by mapping with nuclease S1), as well as protein binding domains (consensus sequences) responsible for the binding of RNA polymerase.

In some embodiments, the promoter is specific to the cell type in which Kir7.1 is to be expressed. For example, suitable cell types including retinal pigment epithelium, small intestinal cells, uterine cells, kidney cells, among others. The promoters may be specific to polarized cells, e.g., cells that have directionality and the Kir7.1 potassium pump plays a role in maintaining the polarization of the cells. Suitable promoters that may be used in a tissue specific manner include the RPE promoters (e.g., EF1a or VMD2) described and the promoters found below in Table 7. In some embodiments, the promoter is active in the retinal pigment epithelium (RPE) in the eye of a subject.

The “promoter” may be the endogenous promoter for the KCNJ13 gene found, for example, in a subject. Alternatively, the promoter may be a heterologous promoter (i.e., a promoter for a non-KCNJ13 gene). Heterologous promoters useful in the practice of the present invention include, without limitation, constitutive, inducible, temporally-regulated, developmentally regulated, chemically regulated, tissue-preferred and tissue-specific promoters.

Suitable heterologous promoters may include, without limitation, an EF1a promoter or a VMD2 promoter. An exemplary EF1a promoter is provided as SEQ ID NO:3. An exemplary VMD2 promoter is provided as SEQ ID NO:4. Suitable EF1a promoters may also include variants of the EF1a promoter provided as SEQ ID NO:3 having at least 60%, 65%, 70%, 75%, 80%, 85%, 90%, 91%, 92%, 93%, 94%, 95%, 96%, 97%, 98%, 99%, or 100% sequence identity to SEQ ID NO:3. Suitable VMD2 promoters may also include variants of the VMD2 promoter provided as SEQ ID NO:4 having at least 60%, 65%, 70%, 75%, 80%, 85%, 90%, 91%, 92%, 93%, 94%, 95%, 96%, 97%, 98%, 99%, or 100% sequence identity to SEQ ID NO:4.

Regarding polynucleotides such the promoters and Kir7.1 polynucleotides described herein, the phrases “% sequence identity,” “percent identity,” or “% identity” refer to the percentage of base matches between at least two polynucleotide sequences aligned using a standardized algorithm. Methods of polynucleotide sequence alignment are well-known.

In some embodiments, the disclosed polynucleotides encoding a Kir7.1 polypeptide are operably connected to the promoter. As used herein, a polynucleotide is “operably connected” or “operably linked” when it is placed into a functional relationship with a second polynucleotide sequence. For instance, a promoter is operably linked to a polynucleotide if the promoter is connected to the polynucleotide such that it may effect transcription of the polynucleotides. In various embodiments, the polynucleotides may be operably linked to at least 1, at least 2, at least 3, at least 4, at least 5, or at least 10 promoters.

As used herein, a “Kir7.1 polypeptide” refers to an inward rectifier potassium channel characterized by a greater tendency to allow potassium to flow into the cell rather than out of it. A human Kir7.1 polypeptide is provided as SEQ ID NO:1. A Kir7.1 polypeptide may also be a variant or homolog of the human Kir7.1 polypeptide provided as SEQ ID NO:1 having at least 60%, 65%, 70%, 75%, 80%, 85%, 90%, 91%, 92%, 93%, 94%, 95%, 96%, 97%, 98%, 99%, or 100% sequence identity to SEQ ID NO:1.

As used herein, the terms “protein” or “polypeptide” or “peptide” may be used interchangeably to refer to a polymer of amino acids. A “polypeptide” as contemplated herein typically comprises a polymer of naturally occurring amino acids (e.g., alanine, arginine, asparagine, aspartic acid, cysteine, glutamine, glutamic acid, glycine, histidine, isoleucine, leucine, lysine, methionine, phenylalanine, proline, serine, threonine, tryptophan, tyrosine, and valine).

Regarding Kir7.1 polypeptides, the phrases “% sequence identity,” “percent identity,” or “% identity” refer to the percentage of residue matches between at least two amino acid sequences aligned using a standardized algorithm. Methods of amino acid sequence alignment are well-known. Some alignment methods take into account conservative amino acid substitutions. Such conservative substitutions, explained in more detail below, generally preserve the charge and hydrophobicity at the site of substitution, thus preserving the structure (and therefore function) of the polypeptide. Percent identity for amino acid sequences may be determined as understood in the art. (See, e.g., U.S. Pat. No. 7,396,664, which is incorporated herein by reference in its entirety). A suite of commonly used and freely available sequence comparison algorithms is provided by the National Center for Biotechnology Information (NCBI) Basic Local Alignment Search Tool (BLAST), which is available from several sources, including the NCBI, Bethesda, Md., at its website. The BLAST software suite includes various sequence analysis programs including “blastp,” that is used to align a known amino acid sequence with other amino acids sequences from a variety of databases.

Polypeptide sequence identity may be measured over the length of an entire defined polypeptide sequence, for example, as defined by a particular SEQ ID number, or may be measured over a shorter length, for example, over the length of a fragment taken from a larger, defined polypeptide sequence, for instance, a fragment of at least 15, at least 20, at least 30, at least 40, at least 50, at least 70 or at least 150 contiguous residues. Such lengths are exemplary only, and it is understood that any fragment length supported by the sequences shown herein, in the tables, figures or Sequence Listing, may be used to describe a length over which percentage identity may be measured.

The Kir7.1 polypeptides disclosed herein may include “variant” polypeptides, “mutants,” and “derivatives thereof.” As used herein the term “wild-type” is a term of the art understood by skilled persons and means the typical form of a polypeptide as it occurs in nature as distinguished from variant or mutant forms. As used herein, a “variant, “mutant,” or “derivative” refers to a polypeptide molecule having an amino acid sequence that differs from a reference protein or polypeptide molecule. A variant or mutant may have one or more insertions, deletions, or substitutions of an amino acid residue relative to a reference molecule. For example, a Kir7.1 polypeptide mutant or variant may have one or more insertions, deletions, or substitution of at least one amino acid residue relative to the Kir7.1 “wild-type” polypeptides disclosed herein. The polypeptide sequence of a “wild-type” Kir7.1 polypeptides is provided as SEQ ID NO:1. This sequence may be used as a reference sequence.

The Kir7.1 polypeptides provided herein may be full-length polypeptides or may be fragments of the full-length polypeptide. As used herein, a “fragment” is a portion of an amino acid sequence which is identical in sequence to but shorter in length than a reference sequence. A fragment may comprise up to the entire length of the reference sequence, minus at least one amino acid residue. For example, a fragment may comprise from 5 to 350 contiguous amino acid residues of a reference polypeptide, respectively. In some embodiments, a fragment may comprise at least 5, 10, 15, 20, 25, 30, 40, 50, 60, 70, 80, 90, 100, 150, or 250 contiguous amino acid residues of a reference polypeptide. Fragments may be preferentially selected from certain regions of a molecule. The term “at least a fragment” encompasses the full length polypeptide. A fragment of a Kir7.1 polypeptide may comprise or consist essentially of a contiguous portion of an amino acid sequence of a full-length Kir7.1 polypeptide (See SEQ ID NO:1). A fragment may include an N-terminal truncation, a C-terminal truncation, or both truncations relative to the full-length Kir7.1 polypeptide.

A “deletion” in a Kir7.1 polypeptide refers to a change in the amino acid sequence resulting in the absence of one or more amino acid residues. A deletion may remove at least 1, 2, 3, 4, 5, 10, 20, 50, 100, 200, or more amino acids residues. A deletion may include an internal deletion and/or a terminal deletion (e.g., an N-terminal truncation, a C-terminal truncation or both of a reference polypeptide).

“Insertions” and “additions” in a Kir7.1 polypeptide refer to changes in an amino acid sequence resulting in the addition of one or more amino acid residues. An insertion or addition may refer to 1, 2, 3, 4, 5, 10, 20, 30, 40, 50, 60, 70, 80, 90, 100, 150, 200, or more amino acid residues. A variant of a Kir7.1 polypeptide may have N-terminal insertions, C-terminal insertions, internal insertions, or any combination of N-terminal insertions, C-terminal insertions, and internal insertions.

The amino acid sequences of the Kir7.1 polypeptide variants, mutants, derivatives, or fragments as contemplated herein may include conservative amino acid substitutions relative to a reference amino acid sequence. For example, a variant, mutant, derivative, or fragment polypeptide may include conservative amino acid substitutions relative to a reference molecule. “Conservative amino acid substitutions” are those substitutions that are a substitution of an amino acid for a different amino acid where the substitution is predicted to interfere least with the properties of the reference polypeptide. In other words, conservative amino acid substitutions substantially conserve the structure and the function of the reference polypeptide. Conservative amino acid substitutions generally maintain (a) the structure of the polypeptide backbone in the area of the substitution, for example, as a beta sheet or alpha helical conformation, (b) the charge or hydrophobicity of the molecule at the site of the substitution, and/or (c) the bulk of the side chain.

The disclosed variant and fragment Kir7.1 polypeptides described herein may have one or more functional or biological activities exhibited by a reference polypeptide (e.g., one or more functional or biological activities exhibited by a wild-type Kir7.1 polypeptide (i.e, SEQ ID NO: 1). Suitably, the disclosed variant or fragment Kir7.1 polypeptide retains at least 20%, 40%, 60%, 80%, or 100% of the potassium conductance properties of the reference polypeptide. As used herein, a “functional fragment” of a Kir7.1 polypeptide is a fragment of, for example, the polypeptide of SEQ ID NO:1 that retains at least 20%, 40%, 60%, 80%, or 100% of the potassium conductance properties of the full-length ADH polypeptide.

Furthermore, it will be readily apparent to a person of ordinary skill in the art that additional Kir7.1 polypeptide variants may be created by aligning Kir7.1 polypeptide sequences from two or more species. Based on these alignments, a person of ordinary skill in the art may identify various amino acid residues that may be altered (i.e, substituted, deleted, etc.) without substantially affecting the potassium conductance properties of the polypeptide. For example, a person of ordinary skill in the art would appreciate that substitutions in a reference Kir7.1 polypeptide could be based on alternative amino acid residues that occur at the corresponding position in other Kir7.1 polypeptides from other species.

In some embodiments, the gene therapy vector may be a lentiviral vector or adeno-associated viral (AAV) vector including a polynucleotide having at least 60%, 65%, 70%, 75%, 80%, 85%, 90%, 91%, 92%, 93%, 94%, 95%, 96%, 97%, 98%, 99%, or 100% sequence identity sequence identity to SEQ ID NO:5 (EF1a-Kir7.1) or SEQ ID NO:6 (VMD2-Kir7.1).

### Therapeutic Compositions

In another aspect, the present invention relates to therapeutic compositions. The therapeutic compositions may include any of the gene therapy vectors described herein and a pharmaceutically-acceptable carrier. The therapeutic compositions may include a pharmaceutically-acceptable carrier, excipient, or diluent, which are nontoxic to the cell or subject being exposed thereto at the dosages and concentrations employed. Often a pharmaceutical diluent is in an aqueous pH buffered solution. Examples of pharmaceutically-acceptable carriers or excipients may include, without limitation, water, buffers such as phosphate, citrate, and other organic acids; antioxidants including ascorbic acid; low molecular weight (less than about 10 residues) polypeptide; proteins, such as serum albumin, gelatin, or immunoglobulins; hydrophilic polymers such as polyvinylpyrrolidone; amino acids such as glycine, glutamine, asparagine, arginine or lysine; monosaccharides, disaccharides, and other carbohydrates including glucose, mannose, or dextrins; chelating agents such as EDTA; sugar alcohols such as mannitol or sorbitol; salt-forming counterions such as sodium; and/or nonionic surfactants such as TWEEN™ brand surfactant, polyethylene glycol (PEG), and PLURONICS™ surfactant.

### Methods of Treatment

In a further aspect of the present invention, methods of treating a subject having a condition associated with insufficient expression or function of a Kir7.1 polypeptide are provided. The methods may include administering a therapeutically effective amount of any one of the gene therapy vectors described herein or any one of the therapeutic compositions described herein to the subject. As used herein, the terms “subject” and “patient” are used interchangeably to refer to both human and nonhuman animals. The term “nonhuman animals” of the disclosure may include mammals and non-mammals, such as nonhuman primates, sheep, dog, cat, horse, cow, pig, mice, rats, and the like. In some embodiments, the subject is a human patient. The subject may be a human patient having cells (i.e., RPE cells) that exhibit insufficient expression or function of a Kir7.1 polypeptide.

Conditions associated with insufficient expression or function of a Kir7.1 polypeptide may include conditions in which a subject has reduced or eliminated Kir7.1 expression or function in or outside a cell as compared to a control. As used herein, a “control” may include subjects having wildtype Kir7.1 function. For example, in some embodiments, a control may be a subject having a wildtype KCNJ13 gene that does not include any loss-of-function mutations in either the non-coding regulatory sequences (i.e., promoter, enhancers, etc.) controlling the expression of the KCNJ13 gene or in the coding region of the KCNJ13 gene (i.e., SEQ ID NOS: 1 and 2).

Subjects may have several “cell” types that may display insufficient expression or function of a Kir7.1 polypeptide. As used herein, a “cell” may refer to cells that normally express a Kir7.1 polypeptide in a wild-type subject. Suitable cells may include, without limitation, eye cells such as retinal cells or retinal pigment epithelium (RPE) cells. Kir7.1 is also expressed in epithelial cells of various organs including kidney, thyroid, CNS neurons, ependymal cells, choroid plexus epithelium, spinal cord, myometrial smooth muscle, small intestine, neural regions of the gastric mucosa as well as gastric parietal cells, and also in the lung, prostate, liver, pancreas, cochlear nucleus, testis and ovaries.

In some embodiments, the condition associated with insufficient expression or function of a Kir7.1 polypeptide may be associated with at least one loss-of-function mutation in a KCNJ13 gene. The human KCNJ13 gene is provided as UniProt 060928. The KCNJ13 gene in other nonhuman subjects may be identified by using homology searching methods well known in the art. Suitable loss-of-function mutations in the KCNJ13 gene may include at least one substitution to the Kir7.1 protein provided as SEQ ID NO:1 selected from the group consisting of W53Ter, Q116R, I120T, T153I, R162Q, R166Ter, L241P, E276A, S105I, and G219Ter. In some embodiments, the condition associated with insufficient expression or function of a Kir7.1 polypeptide may be, without limitation, Leber Congenital Amaurosis 16 (LCA16), retinitis pigmentosa, or Snowflake Vitreoretinal Degeneration (SVD).

In some embodiments, the cell that displays insufficient expression or function of a Kir7.1 polypeptide is within the small intestine of the subject. Suitable vectors may be constructed using a small intestine specific promoter, including, but not limited to, for example, HIFABP, HMUC2, or HLY (found in Table 7) to target the Kir7.1 to the small intestine. Methods of treating a subject with insufficient expression or function of Kir7.1 in the small intestine are provided. The method may include administering a therapeutically effective amount of a gene therapy vector comprising a small intestine specific promoter, e.g., HIFABP, HMUC2, or HLY operably linked to the Kir7.1 polynucleotide or a therapeutic composition comprising the vector to the subject in order to provide expression of Kir7.1 in the small intestine of the subject.

In some embodiments, the cell the displays insufficient expression or function of a Kir7.1 polypeptide within the uterus of a subject. Suitable vectors may be constructed using a smooth muscle specific promoter, for example, SM22a (found in Table 7) to target the Kir7.1 to the uterus. Methods of treating a subject with insufficient expression or function of Kir7.1 in the uterus are provided. The method may include administering a therapeutically effective amount of a gene therapy vector comprising a smooth muscle or uterus specific promoter, e.g., SM22a operably linked to the Kir7.1 polynucleotide or a therapeutic composition comprising the vector to the subject in order to provide expression of Kir7.1 in the uterus. This method may allow for controlling uterine contractions by regulating Kir7.1 expression and/or regulating the potassium balance within smooth muscles of the uterus.

In some embodiment, the cell that displays insufficient expression or function of a Kir7.1 polypeptide is within the kidney of a subject. Suitable promoters that result in kidney specific expression include, but are not limited to, for example, KAP (kidney androgen-regulated protein or NPHS2 (podocin) promoter (See Table 7). Methods of treating a subject having a condition associated with insufficient expression or function of a Kir7.1 polypeptide within the kidney are provided. The methods may include administering a therapeutically effective amount of a gene therapy vector comprising a kidney specific promoter (e.g., KAP or NPHS2) operably linked to the Kir7.1 polynucleotide sequence or a therapeutic composition comprising such vector to the subject in order to express Kir7.1 in the kidney of the subject.

TABLE 7
Promoters specific to cell types.

| | | GenBank Accession No: | |
|---|---|---|---|
| Organ | Promoter | Primers for | Reference |
| Small | HIFABP | NG_011444 | Identification of an intestine- |
| Intestine | (human | (Primers to amplify promoter by PCR for cloning) | specific promoter and inducible |
| | intestinal | A: 5′- | expression of bacterial |
| | fatty acid | CCGCTCGAGTACCTTCCAAGTGCTGTCAAAC-3′ | α-galactosidase in mammalian |
| | binding | (SEQ ID NO: 11) | cells by a lac operon system. |
| | protein | S: 5′-CGACGCGTCATGCTGAATTCCTTAATTTGC- | Ya-Feng et. al. J Anim Sci |
| | promoter) | 3′ (SEQ ID NO: 12) | Biotechnol. 2012 |
| | HMUC2 | U67167 | |
| | (human | S: 5′-CTAGCTAGCTCCTCCCAGCGTAACGTGAGC- | |
| | mucin-2 | 3′- | |
| | promoter) | (SEQ ID NO: 13) | |
| | HLY | A: 5′-GAAGATCTCTAGTGGCAGCCCCATGGTG-3′- | |
| | (human | (SEQ ID NO: 14) | |
| | lysozyme | NM_000239 | |
| | promoter) | S: 5′- | |
| | | CTAGCTAGCCTGTCCTCTTAGGCAGATACAGA- | |
| | | 3′ (SEQ ID NO: 15) | |
| | | A: 5′- | |
| | | GAAGATCTAGAGCCTTCATGTTGACTGCTA-3′ | |
| | | (SEQ ID NO: 16) | |
| Uterus | SM22a | Z68618 | Temporally controlled somatic |
| | | | mutagenesis in smooth muscle. |
| | | | Kühbandner et. al. Genesis. 2000 |
| Kidney | KAP | 5′-flanking region of the KAP gene (−1542 to −466) | The kidney androgen-regulated |
| | (kidney | 16483 | protein promoter confers renal |
| | androgen- | NPHS2 gene (GenBank accession number AF487463 | proximal tubule cell-specific and |
| | regulated | Sequencing of NPHS2 promoter from −628 to ATG was | highly androgen-responsive |
| | protein) | done by PCR two primers: | expression on the human |
| | NPHS2 | forward 5′-GAAAGTTGGGGATGAGGCGA-3′; (SEQ | angiotensinogen gene in |
| | (podocin) | ID NO: 17) | transgenic mice. |
| | | reverse 5'- | Ding Y et. al. J Biol Chem. 1997 |
| | | CAATCAAAGCTTCCTCAGAGCTGCCGGGCGGCT- | Rare functional variants of |
| | | 3′. (SEQ ID NO: 18) | podocin (NPHS2) promoter in |
| | | | patients with nephrotic |
| | | | syndrome. |
| | | | Oleggini et. al. Gene Expr. 2006 |

“Treating” the condition associated with insufficient expression or function of a Kir7.1 polypeptide includes, without limitation, increasing the levels of functional Kir7.1 polypeptide in or outside a cell in a subject. It would be understood by one skilled in the art that an increase in the amount of functional Kir7.1 may only need to be an increase of at least about 10%, preferably at least about 20%, alternatively about 30%, which may result in the proper functioning of the potassium channel within the cell in which it is expressed, leading to alleviation of one or more symptoms of the disease. For example, the ratio of functional to non-functional Kir7.1 within the cell needs to be sufficient to allow for proper functioning of the potassium channel, and may vary depending of cell type and location.

A “therapeutically effective amount” or an “effective amount” as used herein means the amount of a composition that, when administered to a subject for treating a state, disorder or condition is sufficient to effect a treatment (as defined above). The therapeutically effective amount will vary depending on the compound, formulation or composition, the disease and its severity and the age, weight, physical condition and responsiveness of the subject to be treated.

The compositions (i.e. gene therapy vectors and/or therapeutic compositions) described herein may be administered by any means known to those skilled in the art, including, without limitation, locally or systemically, including, for example, intraocularly, topically, intranasally, intramuscularly, or subcutaneously. When administered intraocularly, in some embodiments, the compositions (i.e. gene therapy vectors and/or therapeutic compositions) may be administered subretinally by, for example, injection to at least one retina of the subject. In the retina, the targeted region for delivery of the compositions (i.e. gene therapy vectors and/or therapeutic compositions) may include the central superior retina or macula.

It will be appreciated that the specific dosage administered in any given case will be adjusted in accordance with the composition or compositions being administered, the disease to be treated or inhibited, the condition of the subject, and other relevant medical factors that may modify the activity of the compositions or the response of the subject, as is well known by those skilled in the art. For example, the specific dose for a particular subject depends on age, body weight, general state of health, diet, the timing and mode of administration, medicaments used in combination and the severity of the particular disorder to which the therapy is applied. Dosages for a given patient can be determined using conventional considerations, e.g., by customary comparison of the differential activities of the compositions described herein and of a known agent, such as by means of an appropriate conventional pharmacological or prophylactic protocol. The maximal dosage for a subject is the highest dosage that does not cause undesirable or intolerable side effects. The number of variables in regard to an individual treatment regimen is large, and a considerable range of doses is expected. The route of administration will also impact the dosage requirements.

The effective dosage amounts described herein refer to total amounts administered, that is, if more than one composition is administered, the effective dosage amounts correspond to the total amount administered. The compositions can be administered as a single dose or as divided doses. For example, the composition may be administered two or more times separated by 4 hours, 6 hours, 8 hours, 12 hours, a day, two days, three days, four days, one week, two weeks, or by three or more weeks.

The compositions (i.e. gene therapy vectors and/or therapeutic compositions) described herein may be administered one or more times to the subject to effectively increase the levels of functional Kir7.1 polypeptide in or outside a cell in a subject. The compositions (gene therapy vectors or therapeutic compositions) may be administered based on the number of copies of the expression construct encoding a Kir7.1 polypeptide delivered to the subject. The subject may be administered between 10⁶ and 10¹⁴, or between 10⁸ and 10¹², or between 10⁹ and 10¹¹, or any range therein copies. In embodiments where the gene therapy vector is a viral vector, the subject may be administered between 10⁶ and 10¹⁴, or between 10⁸ and 10¹², or between 10⁹ and 10¹¹, or any range therein viral genomes.

The present disclosure is not limited to the specific details of construction, arrangement of components, or method steps set forth herein. The compositions and methods disclosed herein are capable of being made, practiced, used, carried out and/or formed in various ways that will be apparent to one of skill in the art in light of the disclosure that follows. The phraseology and terminology used herein is for the purpose of description only and should not be regarded as limiting to the scope of the claims. Ordinal indicators, such as first, second, and third, as used in the description and the claims to refer to various structures or method steps, are not meant to be construed to indicate any specific structures or steps, or any particular order or configuration to such structures or steps. All methods described herein can be performed in any suitable order unless otherwise indicated herein or otherwise clearly contradicted by context. The use of any and all examples, or exemplary language (e.g., “such as”) provided herein, is intended merely to facilitate the disclosure and does not imply any limitation on the scope of the disclosure unless otherwise claimed. No language in the specification, and no structures shown in the drawings, should be construed as indicating that any non-claimed element is essential to the practice of the disclosed subject matter. The use herein of the terms “including,” “comprising,” or “having,” and variations thereof, is meant to encompass the elements listed thereafter and equivalents thereof, as well as additional elements. Embodiments recited as “including,” “comprising,” or “having” certain elements are also contemplated as “consisting essentially of” and “consisting of” those certain elements.

Recitation of ranges of values herein are merely intended to serve as a shorthand method of referring individually to each separate value falling within the range, unless otherwise indicated herein, and each separate value is incorporated into the specification as if it were individually recited herein. For example, if a concentration range is stated as 1% to 50%, it is intended that values such as 2% to 40%, 10% to 30%, or 1% to 3%, etc., are expressly enumerated in this specification. These are only examples of what is specifically intended, and all possible combinations of numerical values between and including the lowest value and the highest value enumerated are to be considered to be expressly stated in this disclosure. Use of the word “about” to describe a particular recited amount or range of amounts is meant to indicate that values very near to the recited amount are included in that amount, such as values that could or naturally would be accounted for due to manufacturing tolerances, instrument and human error in forming measurements, and the like. All percentages referring to amounts are by weight unless indicated otherwise.

No admission is made that any reference, including any non-patent or patent document cited in this specification, constitutes prior art. In particular, it will be understood that, unless otherwise stated, reference to any document herein does not constitute an admission that any of these documents forms part of the common general knowledge in the art in the United States or in any other country. Any discussion of the references states what their authors assert, and the applicant reserves the right to challenge the accuracy and pertinence of any of the documents cited herein. All references cited herein are fully incorporated by reference in their entirety, unless explicitly indicated otherwise. The present disclosure shall control in the event there are any disparities between any definitions and/or description found in the cited references.

Unless otherwise specified or indicated by context, the terms “a”, “an”, and “the” mean “one or more.” For example, “a protein” or “an RNA” should be interpreted to mean “one or more proteins” or “one or more RNAs,” respectively.

The following examples are meant only to be illustrative and are not meant as limitations on the scope of the invention or of the appended claims.

### EXAMPLES

### Example 1—A Precision Medicine Cure for Mutation-Specific Blindness

Leber Congenital Amaurosis (LCA) is an inherited pediatric blindness that is associated with at least 21 different genes. We used patient-derived iPSC-RPE cells to reveal the molecular mechanisms underlying LCA16, which is due to a nonsense mutation in the KCNJ13 gene resulting in a nonfunctional Kir7.1 ion channel. Using either read-through or gene augmentation, we rescued Kir7.1 channel function in patient-derived iPSC-RPE cells via a precision medicine approach.

Mutations in at least 21 genes that are expressed in the outer retinal photoreceptors and retinal pigment epithelium (RPE) cause a form of inherited blindness known as Leber Congenital Amaurosis (LCA), from birth and early childhood. Within the last decade, autosomal recessive mutations in the KCNJ13 gene (603203 on chromosome locus 2q37.1) have been identified in patients with an LCA phenotype (LCA16 OMIM-614186, the 16^{th} gene shown to cause LCA)¹⁻³. LCA16 pathogenic allelic variants include c.158G>A (p.Trp53Ter), c.359T>C (p.Iso120Thr), c.458C>T (p.Thr153Iso), c.496C>T (p.Arg166Ter), and c.722T>C (p.Leu241Pro)^{1,2,4}. In addition, the compound heterozygous KCNJ13 mutations c.314 G>T (p.Ser105Iso) and c.655C>T (p.G219Ter) are known to cause early-onset retinal dystrophy in an LCA patient⁵. An autosomal dominant KCNJ13 mutation, c.484C>T (p.Arg162Trp), causes early-onset blindness called snowflake vitreoretinal degeneration (SVD OMIM-193230)⁶.

Advances in genetic screening will undoubtedly improve our understanding of the array of disorders caused by channelopathies and expand our understanding of the role that KCNJ13 plays in health and disease. An inwardly rectifying potassium channel, Kir7.1, is encoded by KCNJ13 and is expressed in several tissues^{7,8}. In the retina, Kir7.1 is expressed exclusively in cell apical processes of RPE, in which it modulates retinal function and health. The role of the Kir7.1 channel in other organs remains to be elucidated^{9,10}.

A loss-of-function in KCNJ13, as with other channelopathies, is a convenient therapeutic target. We adopted a precision medicine approach in which we used patient-derived iPSC-RPE cells to model LCA16 and explore novel therapies based on mutation-specific and gene-augmentation approaches.

### Results

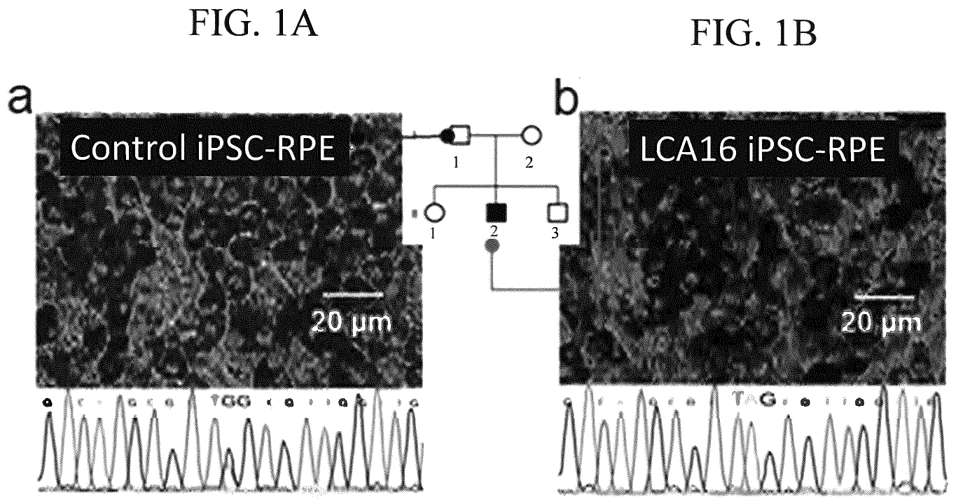

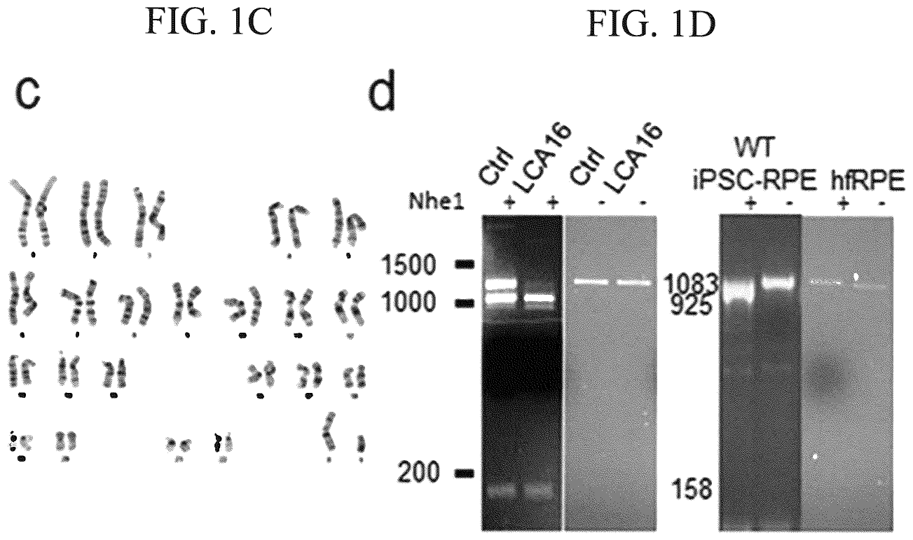

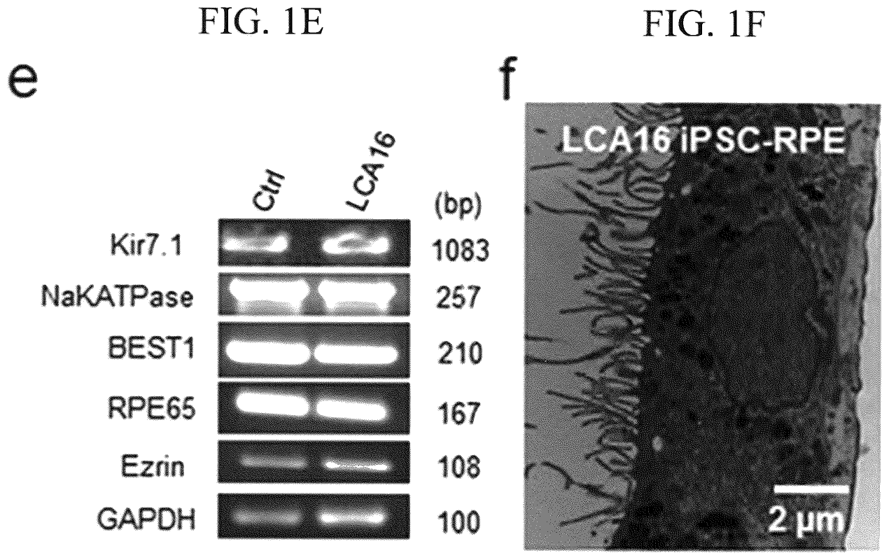

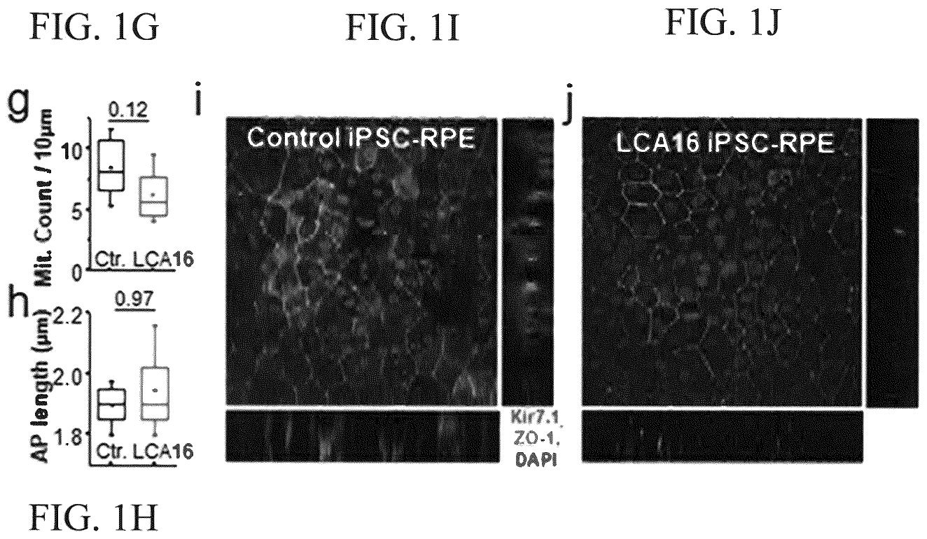

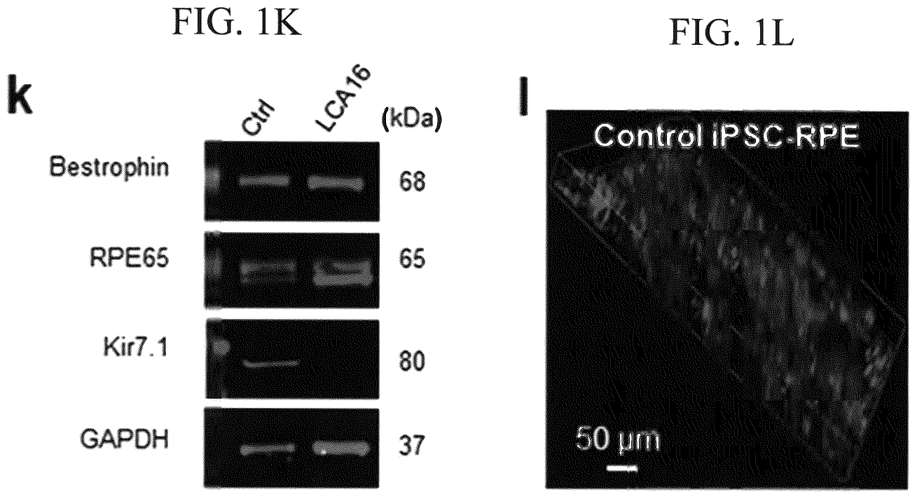

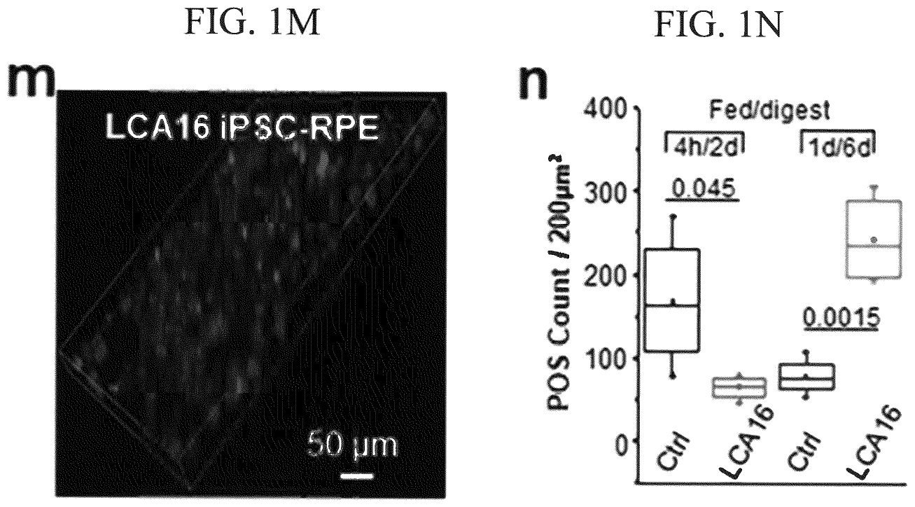

We have previously reported that targeted inhibition of Kir7.1 in the mouse retina (induced using either siRNA or a pharmacological blocker) causes an altered electroretinogram phenotype, consistent with that observed in LCA16 patients¹¹. Here, we outline our development of patient-derived iPSC-RPE cells from skin biopsies from one LCA16 patient carrying a nonsense mutation (Trp53Ter) in exon 2 of the KCNJ13 gene, and an unaffected healthy family member. We were able to model characteristic LCA16 pathological features in RPE cells obtained via in vitro differentiation using a cocktail of transcription factors¹². These cells had normal RPE morphology, including a cobblestone appearance and pigmentation (FIGS. 1A and 1B). DNA sequencing confirmed that the control cells were heterozygous while the LCA16 cells were homozygous for the mutation 158G>A. In addition, the LCA16 cells had a normal karyotype (FIG. 1C). The LCA16 mutation introduced a restriction site for Nhe1, enabling the Kir7.1 mutant sequence to be identified in patient-derived iPSC-RPE, further verifying the presence of a homozygous mutation (FIG. 1D). The control iPSC-RPE were tested and found to be heterozygous carriers, consistent with the genotype of the donor (FIG. 1D). There was no difference in the expression of RPE-specific genes between the two cell types (FIG. 1E). Thus, the patient-derived iPSC-RPE conformed with the genotype of inherited retinal dystrophy and therefore provided a disease-specific cellular model¹.

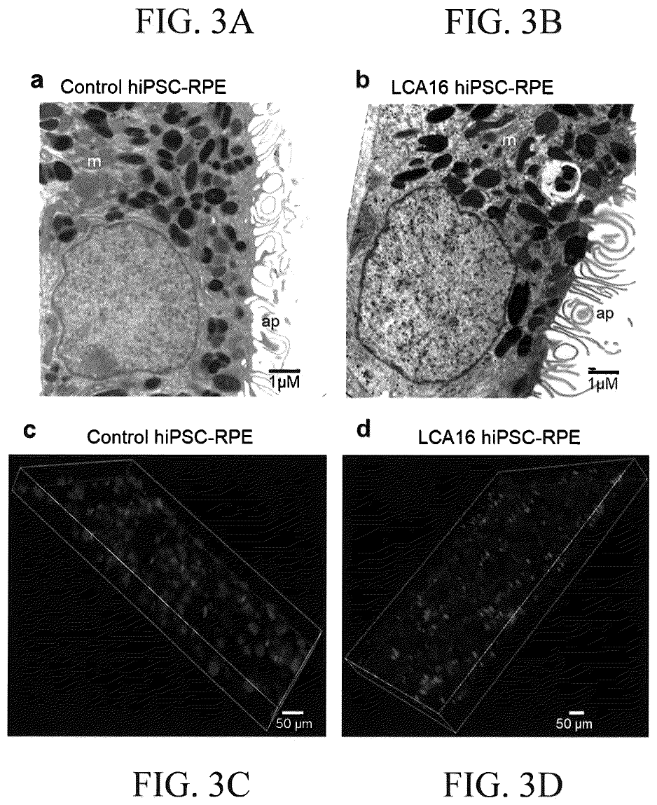

Kir7.1 channels are localized within the highly specialized apical membrane processes of the RPE^{11,13}. Electron microscope image analysis of intact apical membrane structures showed that the cells had a polarized structure, including intact basal membrane in-foldings and elongated apical processes that measured 1.49±0.05 μm in length in the controls, and 1.5±0.14 μm in length in the mutants (P=0.96, n=7) (FIGS. 1F, H and FIGS. 3A-3D). The distribution and number of mitochondria in the two cell lines appeared normal, averaging 8.4±1 and 6.22±0.8 in the control and mutant cells, respectively (P=0.12, n=6) (FIGS. 1F and 1G). Kir7.1 protein expression was detected on the apical membrane of mature control iPSC-RPE cells but not in LCA16 iPSC-RPE cells (FIGS. 1I and 1J). We did not find any difference in protein expression between the two cell lines, except for Kir7.1 (FIG. 1K). The Trp53Ter locus is located within the second exon of the 3-exon KCNJ13 sequence. We have previously shown that a nonsense substitution at amino acid 53 results in a truncated protein product, which explains why the LCA16 patient-derived iPSC-RPE failed to express the full-length Kir7.1 protein.

One of the key physiological functions of RPE cells is the daily phagocytosis of the photoreceptor outer segment, which contributes to the renewal process. To test whether the absence of normal Kir7.1 protein alters phagocytosis, we fed both control and LCA16 iPSC-RPE cell cultures with fluorescently labeled photoreceptor outer segments (POS). The cells were fed the POS for 4 hrs, and then, phagosome digestion by RPE cells was allowed for an additional 48 hrs. We then determined that control iPSC-RPE showed a higher rate of phagosomal uptake than LCA16 patient-derived iPSC-RPE (169±40 vs 66.5±7.4, P=0.04, n=4) (FIGS. 1L, 1M and 1N). In contrast, when cells were fed with POS for 1 day and then allowed to digest phagosomes for 6 days, the LCA16 iPSC-RPE cells failed to digest the POS (80.2±11.1 vs 244.2±27.6 counts within a 200 μm2 field, P=0.001, n=4) (FIGS. 3A-3D). This finding suggests that the pigmentation observed in LCA16 is likely due to an inability to normally phagocytose POS, which therefore accumulate over time in the retinas of affected individuals.

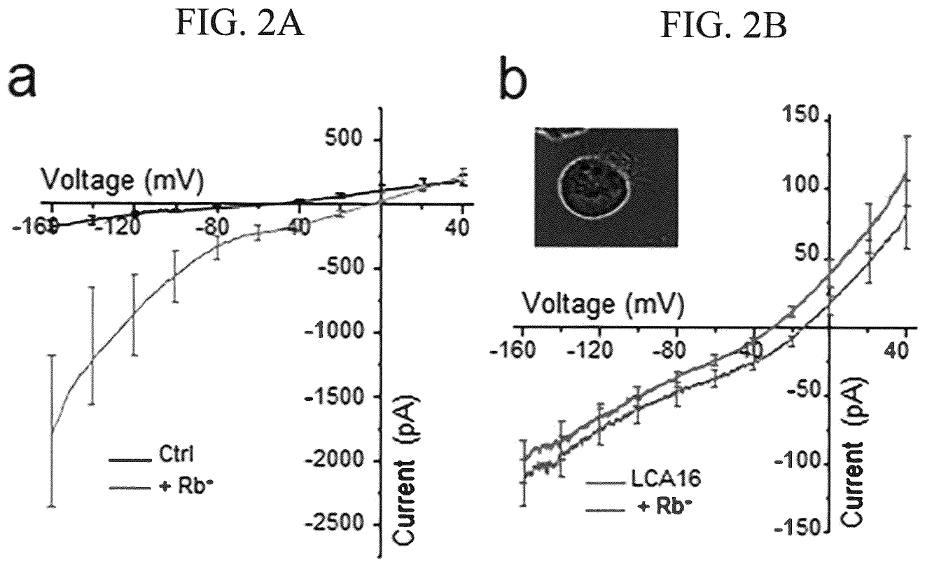

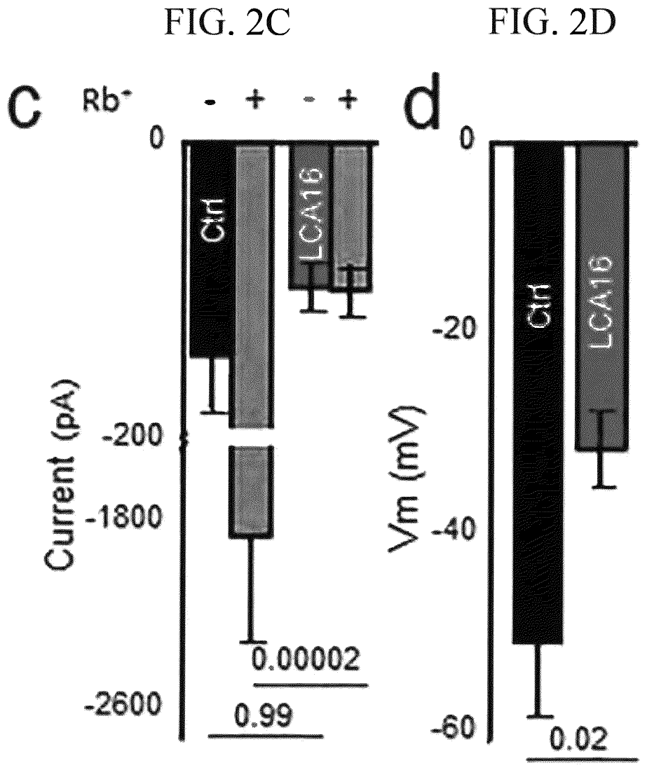

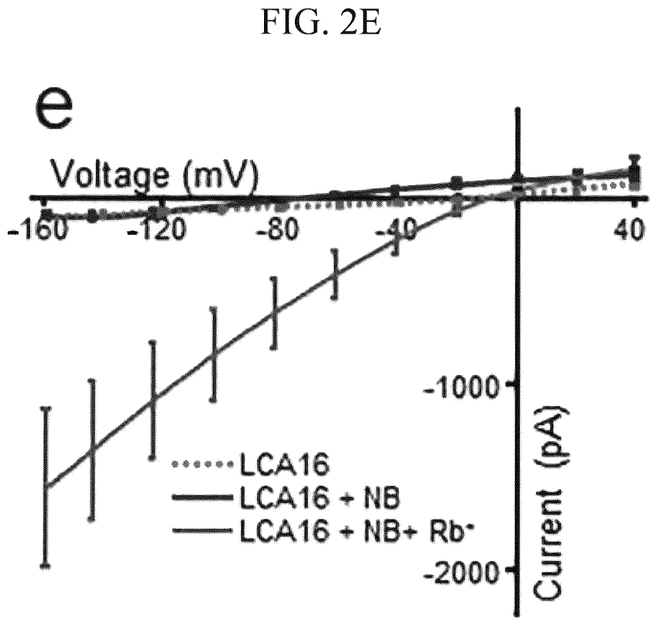

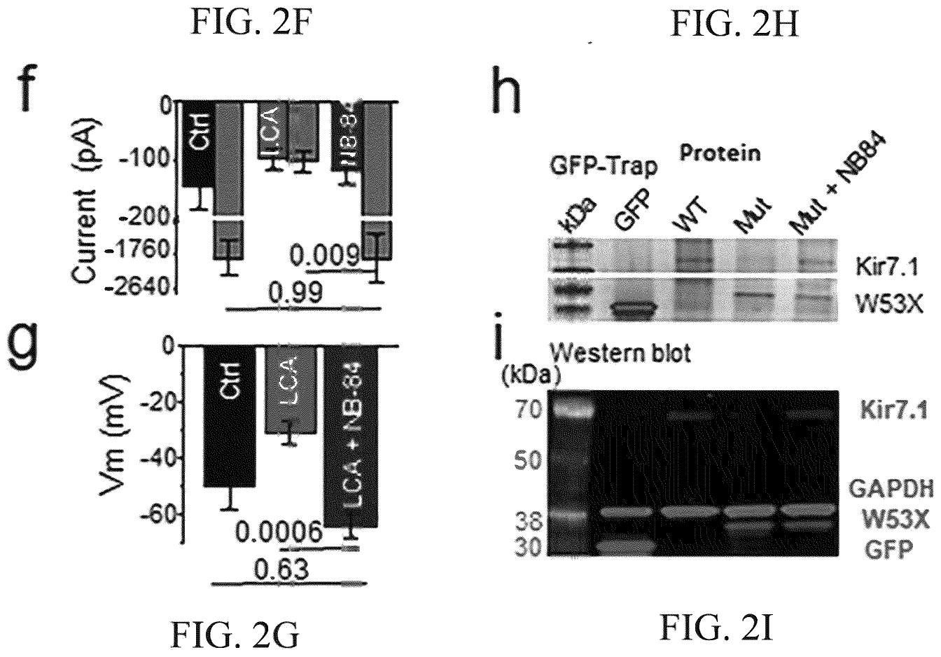

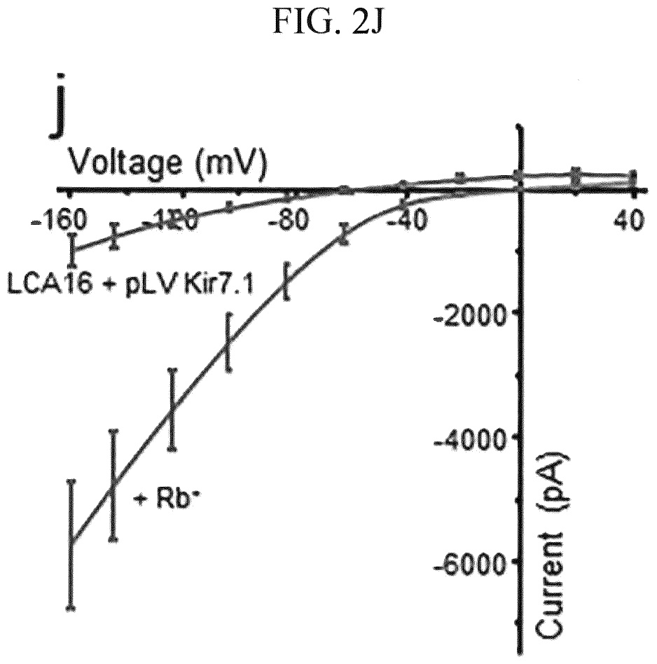

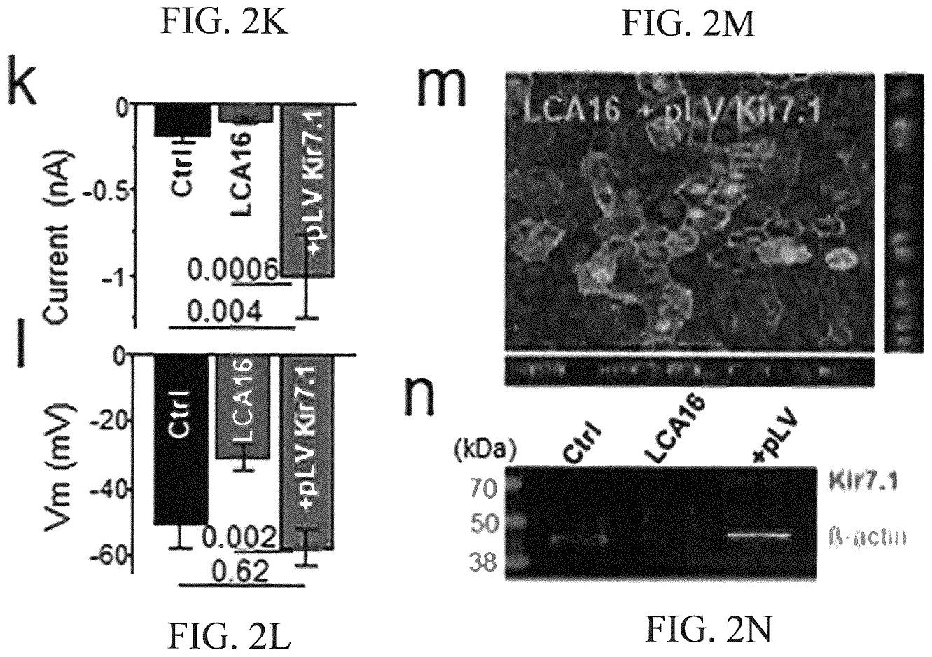

We hypothesized that a nonfunctional channel contributes to the LCA16 phenotype, and we tested this hypothesis by performing whole-cell electrophysiology with iPSC-RPE cells. One of the challenges in studying ion-channels in iPSC cells is their low level or lack of expression. As we have shown the development of specialized apical processes and demonstrated that Kir7.1 localizes to the apical membrane, we were able to detect a small but measurable Kir7.1 current (−120.2±37 pA) in control iPSC-RPE cells. Normal function was confirmed by a fold increase in Rb+ permeability (−439.5±155.7 pA, n=5) (FIGS. 2A and 2C), which is a specific property of the Kir7.1 channel¹⁴. However, in LCA16 iPSC-RPE cells, we did not detect any fold change in the current amplitude mediated by Rb+ conductance (−98.1±15.7 pA & −100.7±15.9 pA, n=9) (FIGS. 2B and 2C). A direct comparison of both current amplitude (P=0.0006 with Rb) and cell membrane potential (−50±5.1 vs −30.6±3.7 mV, P=0.0005; as shown in FIG. 2D) supported our hypothesis that the cause of blindness is a truncated nonfunctional Kir7.1 channel. We have shown earlier using mice and exogenous expression of Kir7.1 channel that a non-functional channel depolarizes RPE cells^{1,11}.

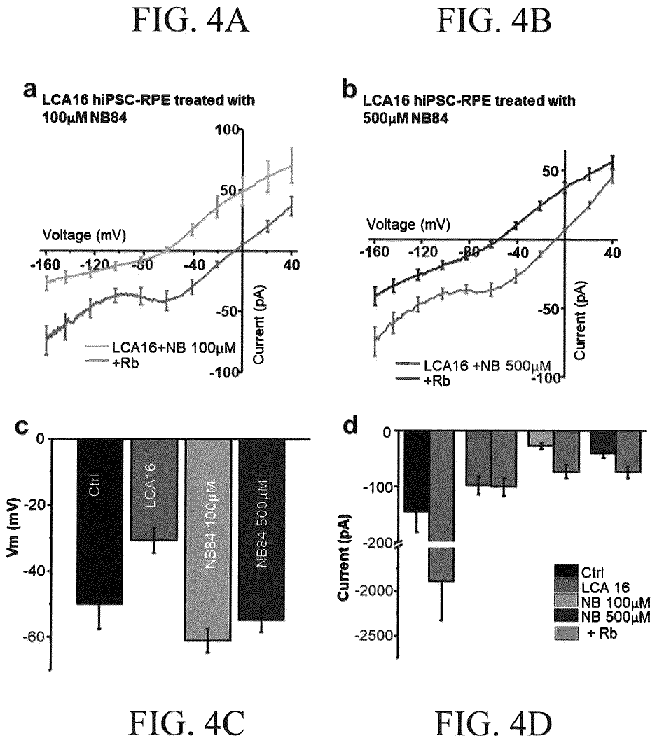

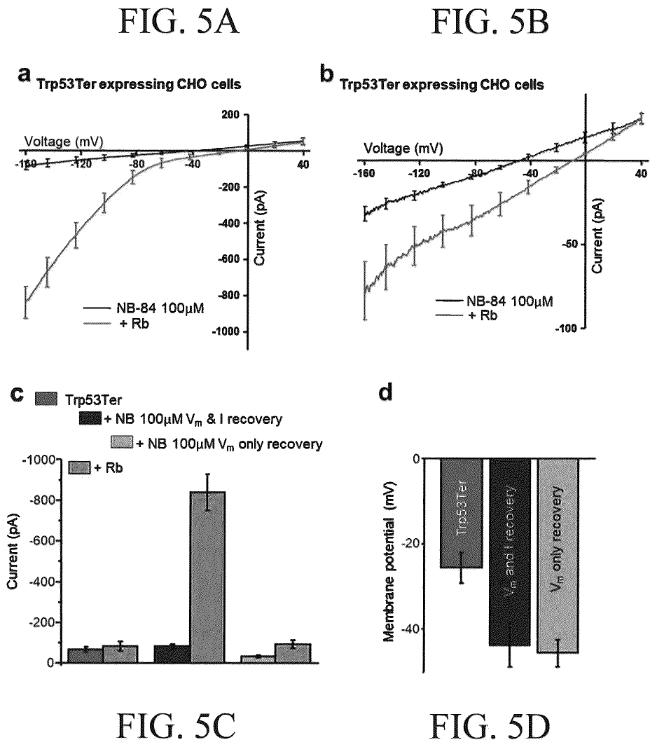

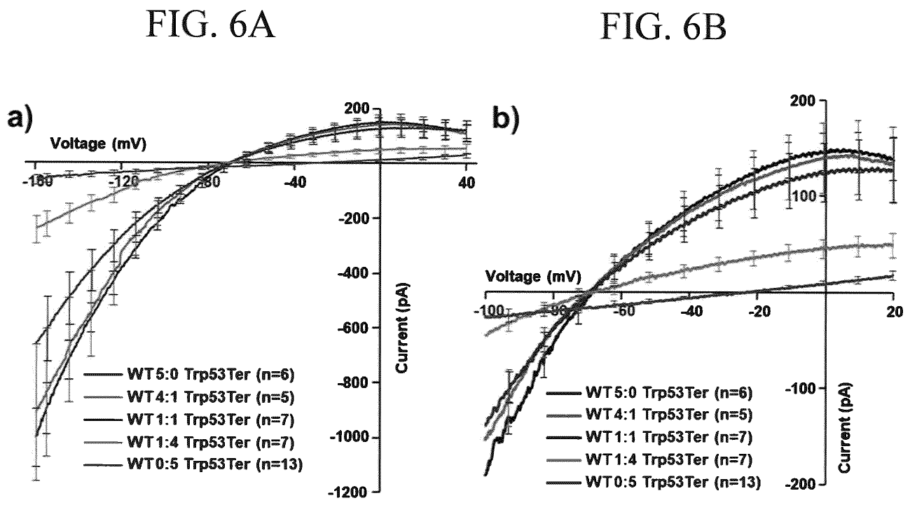

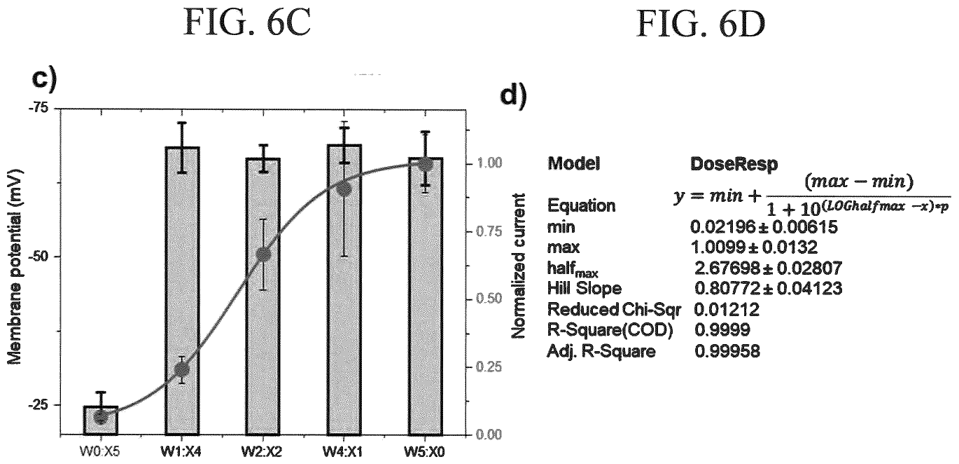

The LCA16 mutation we are studying is a tryptophan (UGG) to amber stop codon (UAG) variant. This nonsense mutation in eukaryotes can be suppressed by the incorporation of near cognate amino acid tRNA in the presence of the small-molecule read-through designer aminoglycoside NB84 (US Patent Publication #20140357590A1)¹⁵⁻¹⁷. We further assessed the functional consequences of NB84-mediated read-through of Kir7.1 current in LCA16 iPSC-RPE cells. Following treatment with 500 μM NB84, we obtained a measurable current in LCA16 iPSC-RPE of −94.3±24 pA that was enhanced by 10-fold upon the introduction of Rb+ (−1562.7±546.7 pA, P=0.005, n=8), a permeant ion (FIGS. 2E and 2F). A significant recovery of membrane potential from −30.6±3.7 in the nontreated cells to −56.3±3.6 mV (P=0.0001, n=10) (FIG. 2G) in the treated cells further justified the use of read-through drug therapy. We were able to determine that a subgroup of cells had rescue in membrane potential without any significant change in current amplitude (FIGS. 4A-4D). This result is perhaps based on which near cognate amino acid (UAG to UAC-tyrosine, UCG-serine, GAG-glutamic acid, or CAG-glutamine) gets incorporated during Kir7.1 protein translation. To optimize the detection of Kir7.1 proteins, we used a stable cell line with a low expression level of a Kir7.1-GFP fusion clone. In NB84-treated cells, a protein band equivalent to the full-length product was detected in addition to the truncated protein band, in conjunction with a rectified membrane potential and current (FIGS. 2H, 2I and FIGS. 5A-5D). NB84 potentiates the specific read-through of a recessive Trp53Ter codon mutation, and we found that as low as a 25% rescue of functional channels was sufficient to circumvent both membrane potential and potassium current and thereby rescue the disease phenotype (FIGS. 6A-6D).

The particular mutation studied herein and other mutations that cause blindness are potential targets for gene therapies given the recent FDA approval of a treatment for blindness^{18,19}. We designed a lentiviral vector with an N-terminal GFP fused to the human Kir7.1 open reading frame under the control of the EF1a promoter²⁰. Intriguingly, after transduction with lentiviral particles Kir7.1-expressing cells presented normal Kir7.1 currents or even slightly higher amplitudes than those observed in the control cells (−920.5±223 pA, P=0.001, n=8). This current was further potentiated by the introduction of Rb+ (−5452.8±929 pA), as expected for a normal functioning Kir7.1 channel (FIGS. 2J and 2K). In addition to K+ currents, the membrane potential of LCA16 iPSC-RPE cells was normalized (−57.5±5.4 mV, P=0.0008) (FIG. 2I). Moreover, newly expressed Kir7.1 was shown to be localized to the apical membranes of diseased iPSC-RPE cells (FIGS. 2M and 2N). Thus, reversal of Kir7.1 function in RPE cells is a potential intervention that will improve vision in patients with congenital blindness due to KCNJ13 mutations.

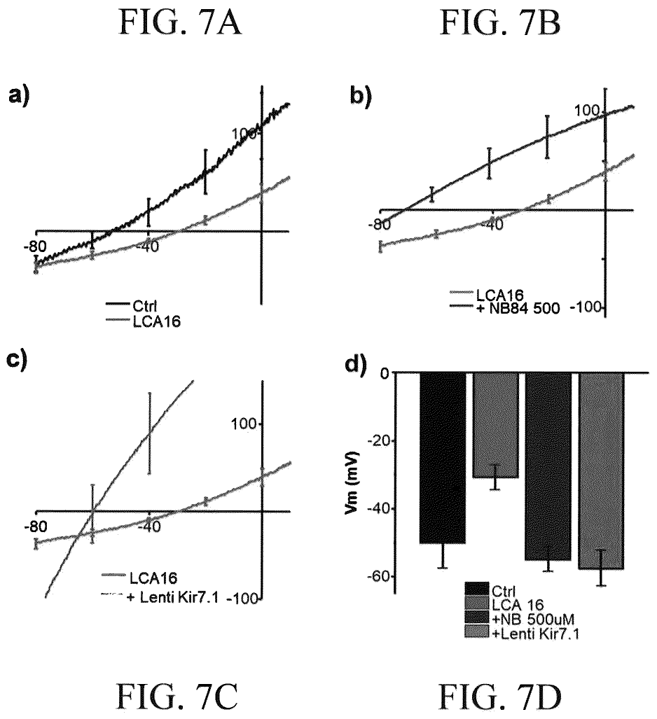

In summary, in autosomal recessive LCA16, we used reprogrammed iPSC-RPE cells to identify unique features associated with a nonsense mutation. The finding that membrane potential was depolarized in diseased cells, which were unable to phagocytose POS, is consistent with the slow progression toward blindness observed in LCA16 patients in addition to their other clinical manifestations, such as electroretinogram abnormalities and retinal pigmentation. Using endogenously expressed Kir7.1 in an iPSC-RPE model, we show that both mutation-specific therapy using nonsense mutation suppression via a designer aminoglycoside and/or the rescue of channelopathy via lentiviral gene augmentation produced a potassium current and normal membrane potential (FIGS. 7A-7D). Thus, we show herein a preclinical therapy for pediatric blindness and a precision medicine approach to a cure for genetic diseases.

### Methods

Differentiation of hiPSC-RPE. Fibroblasts from two subjects were reprogrammed to induced pluripotent stem cells and cultured using established methods¹⁻³. One of the subjects was an LCA16 patient with two copies of the Trp53Ter autosomal recessive mutation in the KCNJ13 gene, and the second subject was heterozygous for this mutation. The hiPSC lines were differentiated to RPE using protocols described earlier²⁻⁵. Briefly, hiPSCs were cultured either on mouse embryonic fibroblasts (MEFs) in iPS cell media (Dulbecco's modified Eagle's medium (DMEM):F12 (1:1), 20% Knockout Serum, 1% minimal essential medium (MEM) non-essential amino acids, 1% GlutaMAX, □-mercaptoethanol, 20 ng/ml FGF-2), or on Matrigel® with mTeSR1 media. Cells were lifted enzymatically and grown as embryoid bodies (EBs) in iPS medium without FGF-2, and at day 4, changed to neural induction medium (NIM; DMEM:F12; 1% N2 supplement, 1% MEM non-essential amino acids, 1% L-Glutamine, 2 μg/ml Heparin), or in mTeSR1 and gradually transitioned to NIM by day 4. There were no differences observed in RPE differentiation between these two approaches. At day 7, free-floating Ebs were plated on laminin-coated culture plates to continue differentiation as adherent culture. At day 16, the 3D neural structures were removed, and medium was switched to retinal differentiation medium (DMEM/F12 (3:1), 2% B27 supplement (without retinoic acid), 1% Antibiotic-Antimycotic). Remaining adhered cells were allowed to continue differentiation for an additional 45 days, followed by microdissection and passaging of pigmented RPE patches to obtain purified monolayers of RPE as described earlier⁵. MEFs, Matrigel® and FGF-2 were purchased from WiCell (Madison, WI), and all other tissue culture reagents were purchased from ThermoFisher. RT-PCR and Restriction Fragment Length Polymorphism (RFLP). Total RNA was isolated from the mature hiPSC-RPE cells from both patient and the carrier using the Rneasy® kit according to manufacturer's instructions (Qiagen). The quality and the concentration of the isolated RNA was measured using a Nanodrop (ThermoFisher) and 200 ng of RNA was used for cDNA synthesis using the Superscript III first strand cDNA synthesis kit according to manufacturer's instructions (ThermoFisher). PCR was performed with MyTaqHS master mix (Bioline) in a final volume of 25 μl with the following conditions: 95 °C for 5 min followed by 35 cycles of denaturation at 95 °C for 15 sec, annealing at 55 °C for 30 sec, and extension at 72 °C for 30 sec. A final extension step was done for 10 min at 72 °C and amplification products were visualized by electrophoresis on a 2% agarose gel containing Midori green advanced stain (Nippon Genetics Europe). For RFLP assay PCR was performed as described with primers specific to the full length KCNJ13 mRNA (Fwd 5′-GCTTCGAATTCCGACAGCAGTAATTG-3′ (SEQ ID NO:7) and Rev 5′-ATCCGGTGGATCCTTATTCTGTCAGT-3′ (SEQ ID NO:8). The PCR products were then digested by Nhe1 restriction enzyme (ThermoFisher) and visualized by electrophoresis on a 2% agarose gel containing Midori green advanced stain (Nippon Genetics Europe).

Transmission Electron Microscopy. Monolayers of hiPSC-RPE on transwell inserts (Corning, Cat #3470) were fixed in a solution of 2.5% glutaraldehyde, 2.0% paraformaldehyde in 0.1M sodium phosphate buffer (PB), pH 7.4 for ~1 hr at room temperature (RT). Samples were rinsed 5×5 minutes in 0.1M PB. The rinsed cultures were then post-fixed in 1% Osmium Tetroxide (OsO₄), 1% potassium ferrocyanide in PB for 1 hr at RT. Following post-fixation, samples were rinsed in PB, as before, followed by 3×5 minute rinses in distilled water to clear the phosphates. The samples were then stained en bloc in uranyl acetate for 2 hrs at RT and dehydrated using ethanol series. The membrane was cut from the transwell support, placed in an aluminum weighing dish, transitioned in propylene oxide (PO) and allowed to polymerize in fresh PilyBed 812 (Polysciences Inc. Warrington, PA). Ultrathin sections were prepared from these polymerized samples and processed before capturing and documenting the images with FEI CM120 transmission electron microscope mounted with AMT BioSprint12 (Advanced Microscopy Techniques, Corp. Woburn, MA) digital camera.

Immunocytochemistry (ICC). Transwell inserts with monolayer of hiPSC-RPE cells from either the patient or control were fixed as follows: the transwell membrane was cut out and fixed by immersing it in 4% paraformaldehyde in phosphate-buffered saline for 10 mins in the dark. The membrane with cells was then washed with chilled PBS twice and blocked for 2 hrs in blocking solution that contained 5% goat serum and 0.25% Tween-20 in 1×PBS. For confocal microscopy, the cells were then incubated for 24-48 hrs with primary antibodies raised against Kir7.1 (mouse monoclonal IgG, 1:250—Santa Cruz), and ZO-1 (rabbit polyclonal, 2.5 μg/ml—ThermoFisher) prepared in incubation solution (Blocking solution diluted in 1:3 with 1×PBS). After incubation with primary antibody, the membranes were washed with chilled 1×PBS thrice and incubated with conjugated secondary antibodies (Donkey anti goat Alexa Fluor® 488, donkey anti Rabbit Alexa Fluor® 594 and DAPI, 1:500) in incubation solution for an hour in dark. A no primary antibody control was included for all experiments. Immunostained samples were imaged on a Nikon C2 confocal microscope (Nikon Instruments Inc., Mellville, NY).

Western blotting. Protein was isolated from >60 day old hiPSC-RPE cells on transwells using Radioimmunoprecipitation assay (RIPA) lysis buffer (ThermoFisher) along with sonication⁶. The protein content of the lysates was measured using commercially available bicinchoninic acid (BCA) assay kit (ThermoFisher). The samples were diluted to contain equal amounts of protein and mixed with 2× Laemmli sample buffer (Bio-Rad) then electrophoresed on NuPAGE® Novex® 4-12% pre-cast polyacrylamide gel (ThermoFisher) followed by transfer to polyvinlidene difluoride (PVDF) membranes using the iBlot® dry blotting system (ThermoFisher). The membranes were blocked with odyssey blocking buffer (LI-COR Biosciences) containing 0.1% Tween-20 for at least 2 hours at 4 °C. After blocking, the membranes were incubated in the appropriate primary antibodies prepared in the Odyssey blocking buffer containing 0.1% Tween-20. The primary antibodies used for this purpose were anti-Kir7.1 (mouse monoclonal, 1:1000—Santa Cruz Biotech), anti-Bestrophin1 (mouse monoclonal, 1:1000—Novus biologicals), anti-RPE65 (mouse monoclonal, 1:1000—ThermoFisher), anti-GFP (mouse monoclonal, 1:1000—NeuroMab), anti-GAPDH (rabbit monoclonal, 1:1000—Cell Signaling), and anti-β-actin (rabbit monoclonal, 1:1000—Cell Signaling Technology) as a loading control. The membranes were incubated with these primary antibodies in combination with control overnight at 4 °C and then washed with Tris buffered saline containing 0.1% Tween-20 4 times before incubating them for another 1 hour with the appropriate IRDye™ secondary antibodies (LI-COR Biosciences) at 1:20000 dilutions in blocking buffer. The membranes were washed 4 times and imaged on an Odyssey® Imaging system.

Photoreceptor Outer Segment (POS) isolation. Fresh bovine eyes were dissected under dim red light and retinas were removed carefully from the eyecup. Isolated retinas were placed in chilled homogenization solution (20% w/v sucrose, 20 mM Tris/Acetate pH 7.2, 2 mM MgCl₂, 10 mM glucose, 5 mM taurine) and mixed gently. The suspension was then passed though gauze to remove clumps. This filtrate was centrifuged through a 25-60% sucrose gradient at 25000 rpm for an hour at 4 °C. The pinkish layer containing the POS was removed and washed with wash solution 1 (20 mM tris acetate pH 7.2 and 5 mM taurine), wash solution 2 (10% sucrose, 20 mM tris acetate pH 7.2 and 5 mM taurine) and wash solution 3 (10% sucrose, 20 mM sodium phosphate pH 7.2 and 5 mM taurine) by centrifuging at 3000 g for 10 mins respectively before resuspending in DMEM containing 2.5% sucrose and stored at −80 °C until use. To fluorescently label the POS, an unlabeled aliquot was thawed and centrifuged at 2400 g for 5 min. The pellet was then re-suspended in 200 μl of DMEM. To this solution 1 μl of WGA (Wheat Germ Agglutinin) conjugated with Alexa Fluor 594® (1 mg/ml, ThermoFisher) was mixed and incubated for 10 min at 37 °C. After completion of incubation with WGA, the tube was again centrifuged at 2400 g for 5 min and the POS pellet was washed twice with DMEM, after which it was used for phagocytosis assays

Phagocytosis Assay. The labelled POS were added to culture media and fed to hiPSC-RPE cells growing in transwells that had a transepithelial electrical resistance (TEER) of >150 Ωcm².¹ The cells were fed POS for either 4 hrs or 24 hrs after which any POS that had not been phagocytosed were removed by washing the cells 3 times with DMEM media. The cells were then incubated for 24 hrs or 6 days respectively before imaging. The images were captured and analyzed with NIS-Elements using a Nikon C2 confocal microscope (Nikon Instruments Inc., Mellville, NY).

Immunoprecipitation of GFP-Fused protein and silver staining: CHO-K1 cells were transiently transfected to exogenously express either the Kir7.1 WT protein or the Kir7.1 Trp53Ter protein as N-terminal fusions with GFP. Cells expressing Trp53Ter protein were then treated with NB848. Immunoprecipitation was performed using GFP-Trap agarose beads (ChromoTek, Germany) according to the manufacturer's protocol.⁶ In brief, the cells were collected and protein isolated as described above for western blotting. GFP-Trap agarose beads were added to the cell lysate and incubated at 4 °C for 2 hours with constant mixing. The mixture was then centrifuged at 2500 g for 2 mins and the beads were washed twice. SDS-sample buffer was added to the beads and incubated at 95 °C for 10 mins followed by centrifugation at 2500×g. The supernatant was separated on a 4-12% acrylamide gel and protein bands were visualized by silver staining using the Pierce Silver Stain kit (ThermoFisher) according to the manufacturer's instructions.

hiPSC-RPE Transduction. Lentivirus, custom engineered to be devoid of pathogenic elements, and carrying KCNJ13 gene fused at N-terminal with green fluorescent protein (GFP) under the control of EF1a promoter, was generated by Cyagen Biosciences (Santa Clara, CA, USA) and used for transduction⁹. LCA-16 hiPSC-RPE monolayer was infected with pLV-EF1α Kir7.1-GFP at an MOI of 200. The cells were cultured for 4-5 days after infection then used for immunocytochemistry and western blotting.

Electrophysiology. Standard whole cell patch clamp on single cells were performed as described⁶ Briefly, the tight monolayer of hiPSC-RPE grown on a 6.5 mm transwell was dissociated into a single cell suspension as follows: the medium in which cells were maintained was completely removed and the cells were washed twice with 0NaCMF solution (135 mM NMDG-Cl, 5 mM KCl, 10 mM HEPES, 10 mM Glucose, 2 mM EDTA-KOH and adjusted to pH 7.4 with NMDG free base). The cells were then incubated with 0NaCMF containing papain (2.5 μl/ml), cysteine (0.3 mg/ml), glutathione (0.25 mg/ml) and taurine (0.05 mg/ml) for 45 mins at 37 °C. The cells were rinsed with 0NaCMF solution to remove enzymes, resuspended in HEPES-Ringer's (HR) solution [NaCl (135 mM), KCl (5 mM), CaCl₂ (1.8 mM), MgCl₂ (1 mM), HEPES (10 mM), D-glucose (10 mM), pH 7.4±0.1 with NaOH, prepared in ddH₂O], and kept on ice for up to 8 hrs until used for electrophysiological recording.

Single hiPSC-RPE cells with distinct apical processes were chosen for conventional patch clamping. Patch pipettes with a resistance of 3-5 mΩ were fabricated from borosilicate capillaries using a pipet puller (P-1000®, Sutter instruments). The glass electrode was then fire polished using a microforge (MF-830®, Narshige). Data acquisition and the holding potential parameters were controlled using the Clampex® software (Axon instruments). Current recorded from the successful patch was amplified using Axopatch 200-B® (Axon Instruments) and filtered at 2 KHz. The signal was digitized using digidata 1400A® (Axon instruments) and analyzed using Clampfit® (Axon Instruments). During patch clamping, HR solution was continuously perfused as an external solution. The patch pipette was filled with solution containing 30 mM KCl, 83 mM K-gluconate, 5.5 mM EGTA-KOH, 0.05 mM CaCl₂, 4 mM MgCl₂, 10 mM HEPES, pH adjusted to 7.2 with KOH and filtered using the 0.2 μm filter.

Statistical analysis. The statistical analysis was performed using Origin (version 9.1) with a two-tailed Student's t-test to assess the significant differences. P<0.05 was considered statistically significant. ANOVA and post hoc Tukey test was also used for multiple comparisons. The data are expressed as the means±SEM.

### References for Methods

1. Singh, R. et al. iPS cell modeling of Best disease: insights into the pathophysiology of an inherited macular degeneration. Hum Mol Genet 22, 593-607 (2013).
2. Meyer, J. S. et al. Modeling early retinal development with human embryonic and induced pluripotent stem cells. Proc Natl Acad Sci USA 106, 16698-703 (2009).
3. Phillips, M. J. et al. Blood-derived human iPS cells generate optic vesicle-like structures with the capacity to form retinal laminae and develop synapses. Invest Ophthalmol Vis Sci 53, 2007-19 (2012).
4. Capowski, E. E. et al. Loss of MITF expression during human embryonic stem cell differentiation disrupts retinal pigment epithelium development and optic vesicle cell proliferation. Hum Mol Genet 23, 6332-44 (2014).
5. Singh, R. et al. Functional analysis of serially expanded human iPS cell-derived RPE cultures. Invest Ophthalmol Vis Sci 54, 6767-78 (2013).
6. Pattnaik, B. R. et al. A Novel KCNJ13 Nonsense Mutation and Loss of Kir7.1 Channel Function Causes Leber Congenital Amaurosis (LCA16). Hum Mutat 36, 720-7 (2015).
7. Parinot, C., Rieu, Q., Chatagnon, J., Finnemann, S. C. & Nandrot, E. F. Large-scale purification of porcine or bovine photoreceptor outer segments for phagocytosis assays on retinal pigment epithelial cells. J Vis Exp (2014).
8. Brendel, C. et al. Readthrough of nonsense mutations in Rett syndrome: evaluation of novel aminoglycosides and generation of a new mouse model. J Mol Med (Berl) 89, 389-98 (2011).
9. Yanez-Munoz, R. J. et al. Effective gene therapy with nonintegrating lentiviral vectors. Nat Med 12, 348-53 (2006).

### References for Example 1 Besides Methods

Pattnaik, B. R. et al. A Novel KCNJ13 Nonsense Mutation and Loss of Kir7.1 Channel Function Causes Leber Congenital Amaurosis (LCA16). Hum Mutat 36, 720-7 (2015).
2. Sergouniotis, P. I. et al. Recessive mutations in KCNJ13, encoding an inwardly rectifying potassium channel subunit, cause leber congenital amaurosis. Am J Hum Genet 89, 183-90 (2011).
3. Perez-Roustit, S. et al. Leber Congenital Amaurosis with Large Retinal Pigment Clumps Caused by Compound Heterozygous Mutations in Konj13. Retin Cases Brief Rep 11, 221-226 (2017).
4. Khan, A. O., Bergmann, C., Neuhaus, C. & Bolz, H. J. A distinct vitro-retinal dystrophy with early-onset cataract from recessive KCNJ13 mutations. Ophthalmic Genet 36, 79-84 (2015).
5. Perez-Roustit, S. et al. Leber Congenital Amaurosis with Large Retinal Pigment Clumps Caused by Compound Heterozygous Mutations in Konj13. Retin Cases Brief Rep (2016).
6. Hejtmancik, J. F. et al. Mutations in KCNJ13 cause autosomal-dominant snowflake vitreoretinal degeneration. Am J Hum Genet 82, 174-80 (2008).
7. Krapivinsky, G. et al. A novel inward rectifier K+ channel with unique pore properties. Neuron 20, 995-1005 (1998).
8. Derst, C. et al. Partial gene structure and assignment to chromosome 2q37 of the human inwardly rectifying K+ channel (Kir7.1) gene (KCNJ13). Genomics 54, 560-3 (1998).
9. McCloskey, C. et al. The inwardly rectifying K+ channel KIR7.1 controls uterine excitability throughout pregnancy. EMBO Mol Med 6, 1161-74 (2014).
10. Ghamari-Langroudi, M. et al. G-protein-independent coupling of MC4R to Kir7.1 in hypothalamic neurons. Nature (2015).
11. Shahi, P. K. et al. Abnormal Electroretinogram after Kir7.1 Channel Suppression Suggests Role in Retinal Electrophysiology. Sci Rep 7, 10651 (2017).
12. Singh, R. et al. iPS cell modeling of Best disease: insights into the pathophysiology of an inherited macular degeneration. Hum Mol Genet 22, 593-607 (2013).
13. Yang, D., Pan, A., Swaminathan, A., Kumar, G. & Hughes, B. A. Expression and localization of the inwardly rectifying potassium channel Kir7.1 in native bovine retinal pigment epithelium. Invest Ophthalmol Vis Sci 44, 3178-85 (2003).
14. Shimura, M. et al. Expression and permeation properties of the K(+) channel Kir7.1 in the retinal pigment epithelium. J Physiol 531, 329-46 (2001).
15. Nudelman, I. et al. Repairing faulty genes by aminoglycosides: development of new derivatives of geneticin (G418) with enhanced suppression of diseases-causing nonsense mutations. Bioorg Med Chem 18, 3735-46 (2010).
16. Goldmann, T. et al. A comparative evaluation of NB30, NB54 and PTC124 in translational read-through efficacy for treatment of an USHIC nonsense mutation. EMBO Mol Med 4, 1186-99 (2012).
17. Ramsden, C. M. et al. Rescue of the MERTK phagocytic defect in a human iPSC disease model using translational read-through inducing drugs. Sci Rep 7, 51 (2017).
18. Bennett, J. et al. Safety and durability of effect of contralateral-eye administration of AAV2 gene therapy in patients with childhood-onset blindness caused by RPE65 mutations: a follow-on phase 1 trial. Lancet 388, 661-72 (2016).
19. Dalkara, D., Goureau, O., Marazova, K. & Sahel, J. A. Let There Be Light: Gene and Cell Therapy for Blindness. Hum Gene Ther 27, 134-47 (2016).
20. White, M., Whittaker, R., Gandara, C. & Stoll, E. A. A Guide to Approaching Regulatory Considerations for Lentiviral-Mediated Gene Therapies. Hum Gene Ther Methods 28, 163-176 (2017).

### Example 2—Kir7.1 Gene-Therapy in Cell Culture Models and In Vivo

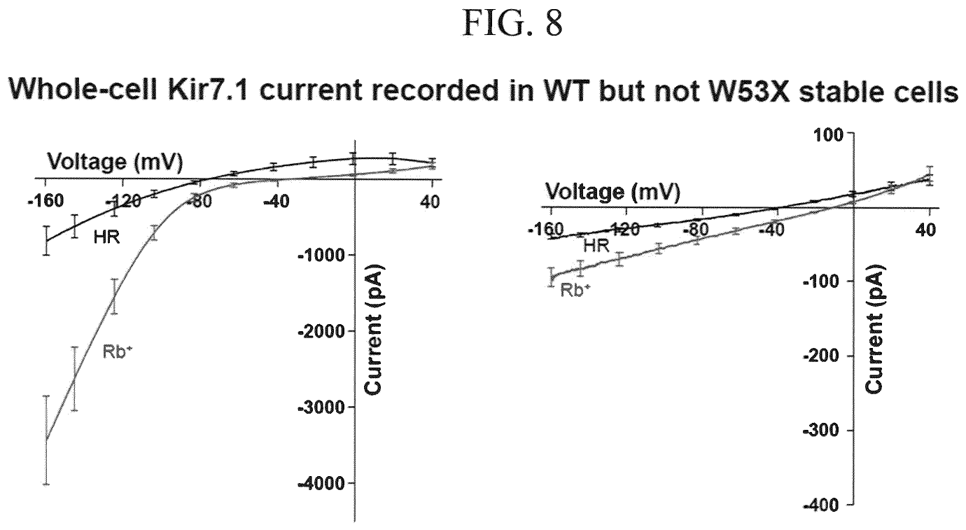

To test the efficacy of gene therapy in a cell culture model of LCA16, we tested the ability of AAV-Kir7.1 to rescue the physiological defects in CHO cells harboring a W53X mutation in the Kcnj 13 gene. FIG. 8 shows whole-cell current voltage relationship from wildtype (left panel) and W53X mutant (right panel) stable cells. Inwardly rectifying K+ current (black trace) in the wildtype stable cell was significantly increased by Rb+ (red trace). In the W53X mutant stable cells on the right, neither K+ nor Rb+ current was recorded (p=1.05E−0.5).

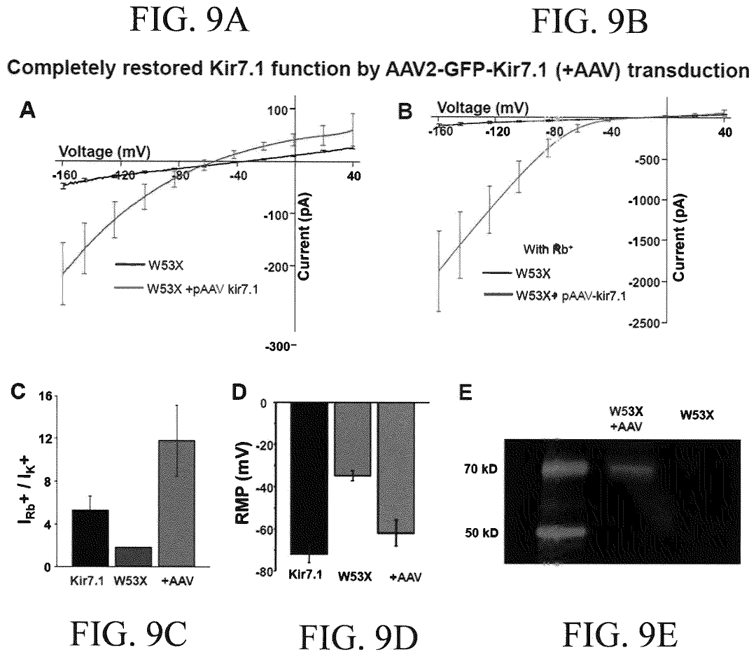

FIG. 9 shows gene augmentation of W53X mutant expressing CHO cells had recovery of average inwardly rectifying K+ current (FIG. 9A. IV plot in red trace) compared to no current before (FIG. 9A. plot in black trace). (FIG. 9B) Average higher Rb+ current (red trace) in W53X mutant expressing cells after gene augmentation. (FIG. 9C) Net increase in Rb+ permeability increased (Blue) through Kir7.1 channel after gene augmentation. (FIG. 9D) Complete recovery of resting membrane potential (RMP) after AAV-Kir7.1 transduction of W53X expressing cells represented as blue box. (FIG. 9E) Western blot results showing expression of full length protein product after gene augmentation in lane W53X+AAV (red band).

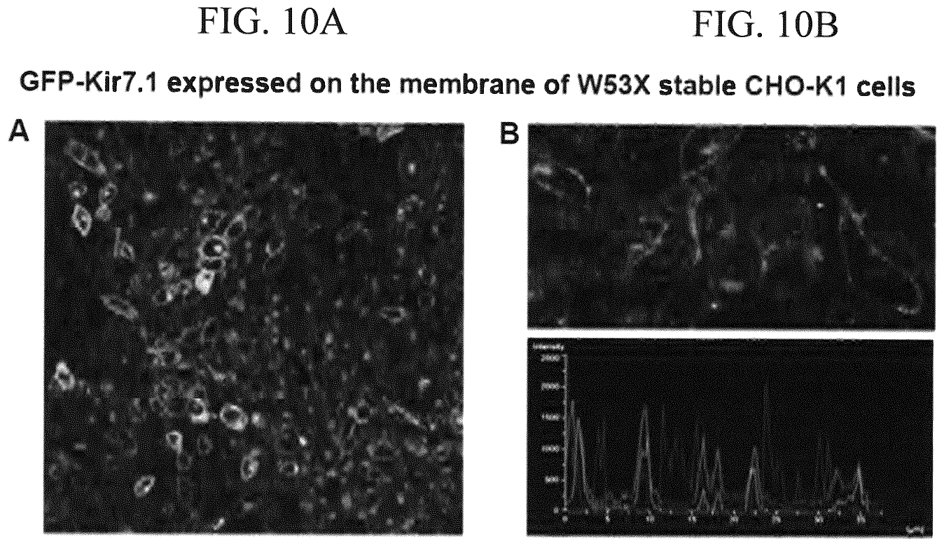

FIG. 10 shows Kir7.1 expression (green) in W53X mutant line after gene augmentation through AAV-Kir7.1 (FIG. 10A). (FIG. 10B) A higher magnification image shows membrane localization of the Kir7.1 protein alongside membrane marker WGA-Alexa 594. In the lower panel is the line scan for red and green showing membrane marker and Kir7.1 co-localize.

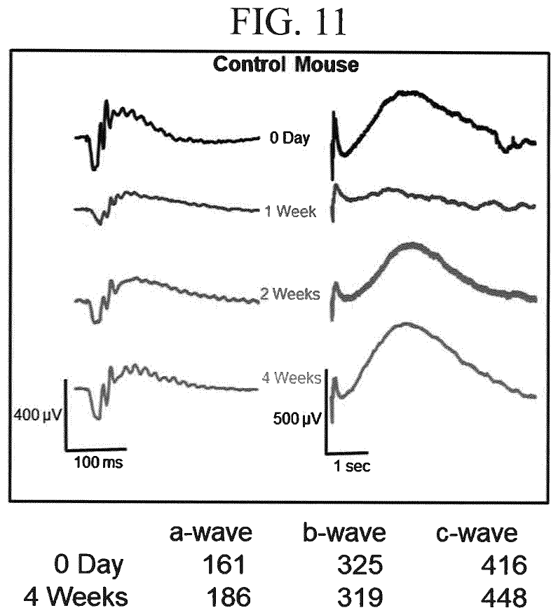

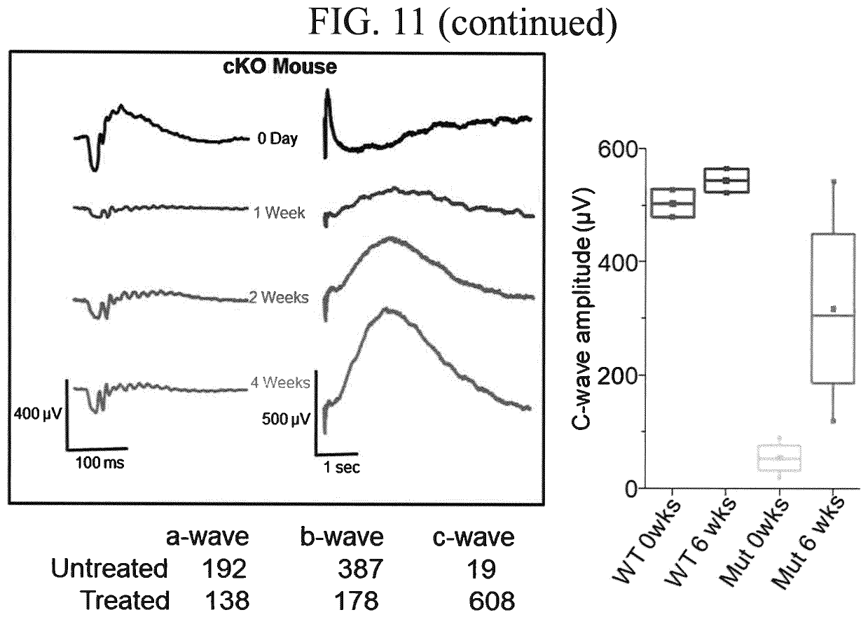

To test the efficacy of gene therapy in vivo, both wild-type and a mouse lacking the Konj13 gene were tested. In FIG. 11 left box is an example of a wild type mouse that received 2 μl of Lenti-EF1a-eGFPKir7.1 by sub-retinal injection. Electrophysiological results are obtained before (black trace) and 1 (blue trace), 2 (red trace), and 4 (green trace) weeks post injection. In FIG. 11 left box, retina responses recorded as normal a- and b-wave are shown on the left and RPE cell response c-wave is shown on the right. Only in the 1^{st} week after injection there was a reduction in retina response otherwise there was hardly any effect of gene therapy on electrophysiological outcome. In FIG. 11 right box we show results from mice lacking Konj13 gene that received 2 μl of Lenti-EF1a-eGFPKir7.1. On the right panel is the RPE response of c-wave, that was completely abolished in these mice (black trace) with slight reduction in a- and b-wave shown in the left panel. Immediately post gene-therapy, we noticed increase in c-wave response starting a week after injection (blue trace on the right panel). Traces show continued increase in c-wave during the following 2 (red trace) and 4 (green) weeks post gene therapy. Average measurements in 4 wild-type and four mice lacking Konj13 gene is shown as box plot with significant recovery in c-wave and no effect on wild-type mice vision. Numbers below the figure shows actual amplitude of a-, b- and c-wave measurements in wild-type and mice lacking Konj13.

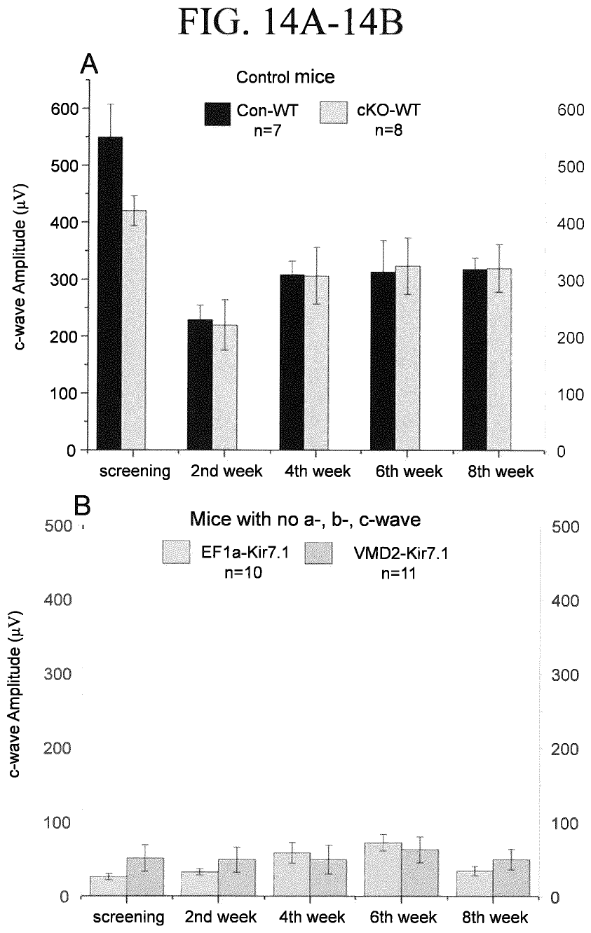

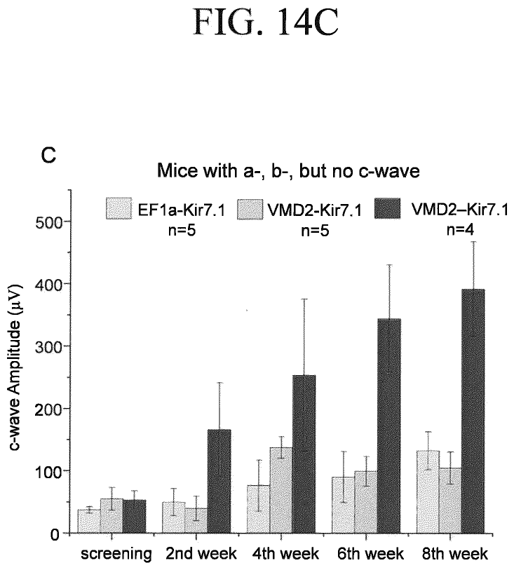

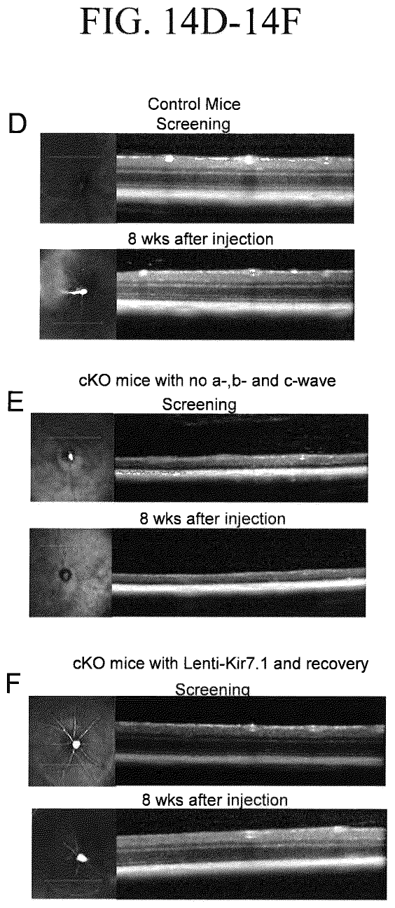

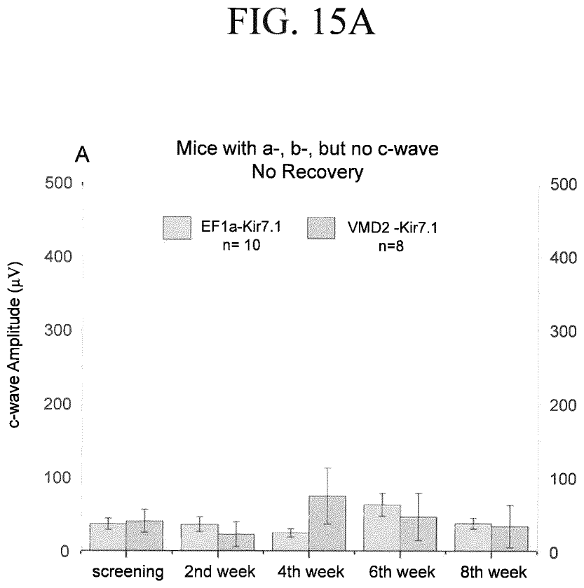

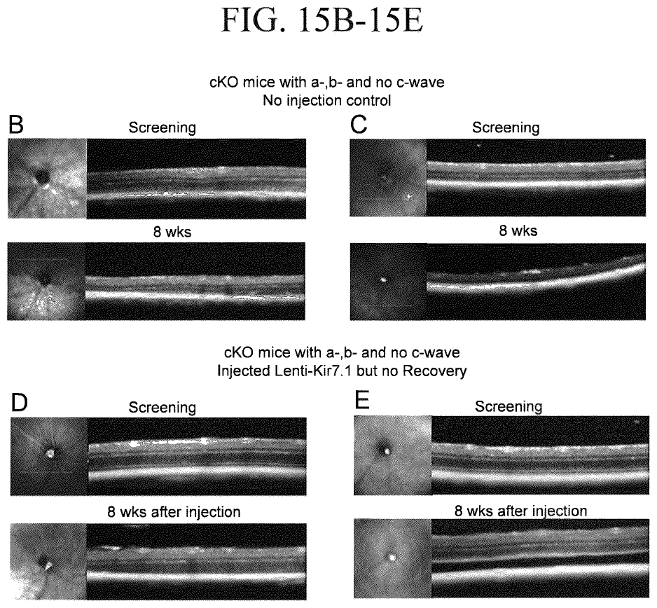

Further, FIGS. 14A-F show functional recovery of Retinal Pigment Epithelial (RPE) cells lacking Kir7.1 protein after gene therapy in the cKO mouse model. FIG. 14A shows injection control on WT mice and the cKO control mice depicting the RPE response functional after 8 weeks with PBS injection. ERG response from the Kir7.1 cKO mice which showed no a-, b- and c-wave during the screening (FIG. 14B). Delivery of the Kir7.1 with lentivirus carrying either constitutive EF1a promoter or RPE specific VMD2 promoter failed to rescue the RPE function due to the severe phenotype as both RPE and photoreceptors were degenerated C-wave from RPE is recovered in the cKO mice, by subretinal delivery of lentivirus carrying kcnj13 gene driven by EF1a and VMD2 promoter, where the photoreceptors were not degenerated but had no response from the RPE cells during screening (FIG. 14C). FIGS. 14D-F show representative optical coherence tomography (OCT) images showing the retinal structure from the control mice, cKO mice (no-a-,b-c-wave) with no recovery and c-wave recovered mice (a-, b- but no-c-wave) during screening and post 8 weeks after lentiviral gene delivery, respectively. Thus, the in vivo data shows that expressing Kir 7.1 in RPE can restore vision in these deficient mice.

### Material and Methods

### Animals

To elucidate the physiological role of KCNJ13 gene in the RPE cells, in vivo, we used a strain that is lacking this gene. Vision in these mice was measured using electroretinography (ERG). The mice were housed and bred at the University of Wisconsin Biotron (Madison, WI)

### Electroretinography

The mice were dark adapted overnight prior to performing ERG. The mice were anesthetized with Ketamine/Xylazine (80:16 mg/kg) cocktail injected intra-peritoneally. While maintaining the body temperature at 37 °C with a heating pad, the pupil of the mouse was dilated with a drop of tropicamide (Bausch+Lomb, Rochester, NY). ERGs were performed using the Espion recording system (Diagnosys) by placing a corneal contact lens (Ocusciences Inc., MO) on the dilated eyes along with Gonak, a 2.5% hypromellose ophthalmic demulcent solution (GONIOVISC, HUB Pharmaceuticals, LLC, CA). A reference and the ground electrode were placed in the mouth and the back respectively. The protocol for ERG consisted of recordings from flash intensities from 0.1 to 30 cd·s·m−2 and 60 Hz line noise was removed using the filter. For c-wave measurements, we used a 5 msec flash of 25 cd·s·m−2 intensity to acquire data during a 5 sec interval. ERG analysis was performed on the mice before and after the sub-retinal injection.

### Sub-Retinal Injection

The KCNJ13 knockout mice with no c-waveforms were used for this purpose. The mice were maintained under tightly controlled temperature (23±5 °C), humidity (40-50%) and light/dark (12/12 h) cycle conditions in 200 lux light environment. Prior to the injection, the mice were anesthetized and pupils were dilated as described above. 2 μl of Lentivirus or Adeno-associated virus (AAV) carrying the functional full length KCNJ13 gene fused with eGFP and driven by EF1a or VMD2 promoters were delivered to the RPE cells through sub-retinal injection using a 10 mm 34 gauge needle. We used a 10 μl Nanofil syringe and UMP3, NanoFil RPE—KIT and Micro4 controller (World precision Instruments, Inc., Sarasota, FL). ERG was performed on these mice at 1 wk, 2 wks, 4 wks and 8 wks post injection and data were analyzed.

### Transgene Expression DETECTION

eGFP fluorescence was detected using confocal microscopy after preparing a flat mount of the isolated RPE. Eyes from the Lentivirus/AAV carrying eGFP-KCNJ13 gene injected mice were retrieved one week post injection. Enucleated eyes from the sacrificed mice were rinsed twice with PBS, a puncture was made at or a serrata with a 28 gauge needle and the eyes were opened along the corneal incisions. The lens was then carefully removed. The eye cup was flattened making incisions radially to the center resulting in a “starfish” appearance. The retina was then separated gently from the RPE layer. The separated RPE and retina were flat mounted on the cover-glass slide and were imaged with NIS-Elements using a Nikon C2 confocal microscope (Nikon Instruments Inc., Mellville, NY). We used 488 nm Diode Lasers for green excitation and images were captured by Low Noise PMT C2 detectors in a Plan Apo VC 20×/0.75, 1 mm WD lens.

### Example 3—Preparation of AAV Viral Vectors for Delivery of Kir7.1 Protein

### AAV Viral Vector Construction

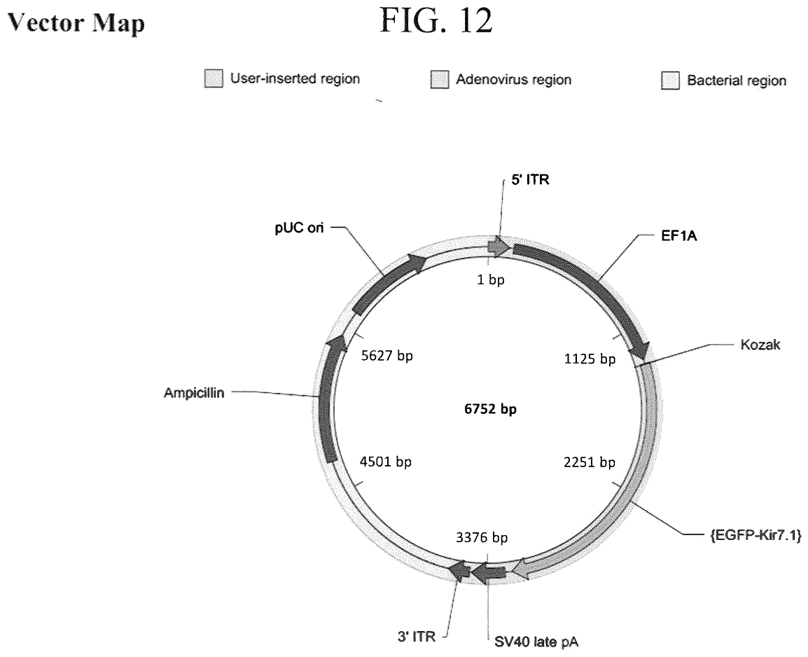

AAV vectors for the delivery of Kir7.1 protein were produced using VectorBuilder software of Cyagen Biosciences and packaging services from Cyagen Biosciences. The following Tables 1-3 and FIG. 12 summarize the construction of AAV vectors that successfully rescued physiological defects in a Kcnj 13 gene.

TABLE 1
Vector Summary

| | |
|---|---|
| Vector ID | VB161122-1168yrz |
| Vector Name (official) | pAAV[Exp]-EF1A>[EGFP-Kir7.1] |
| Date Created (Pacific Time) | 2016 Nov. 22 |
| Size | 6752 bp |
| Vector Type | Adeno-associated virus gene expression vector |
| Inserted Promoter | EF1A |
| Inserted ORF | [EGFP-Kir7.1] |
| Copy Number | High |
| Bacterial Resistance | Ampicillin |
| Cloning Host | Stbl3 |

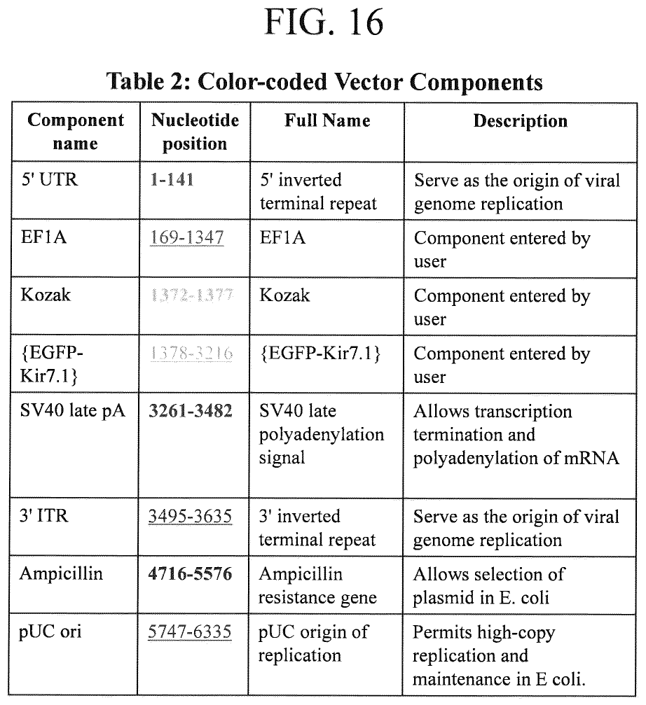

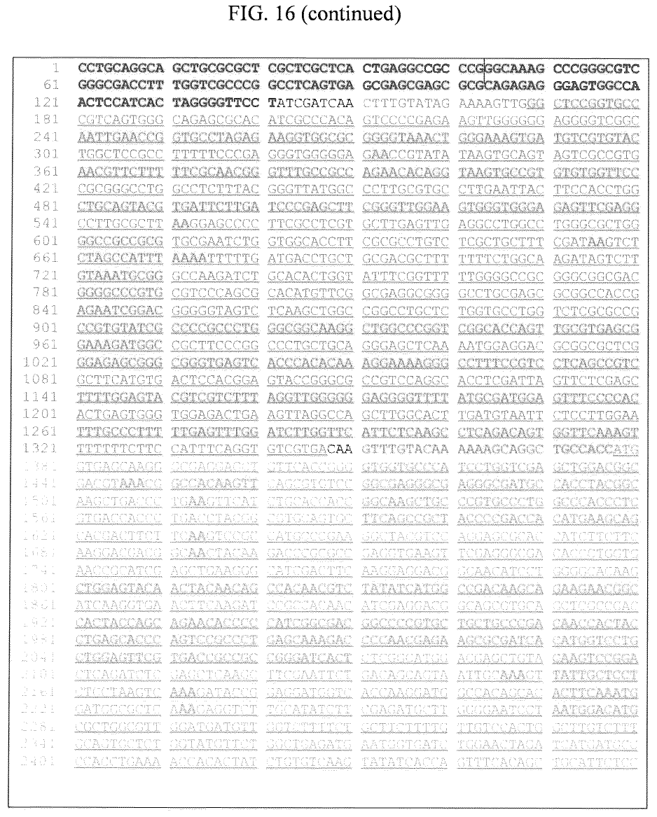

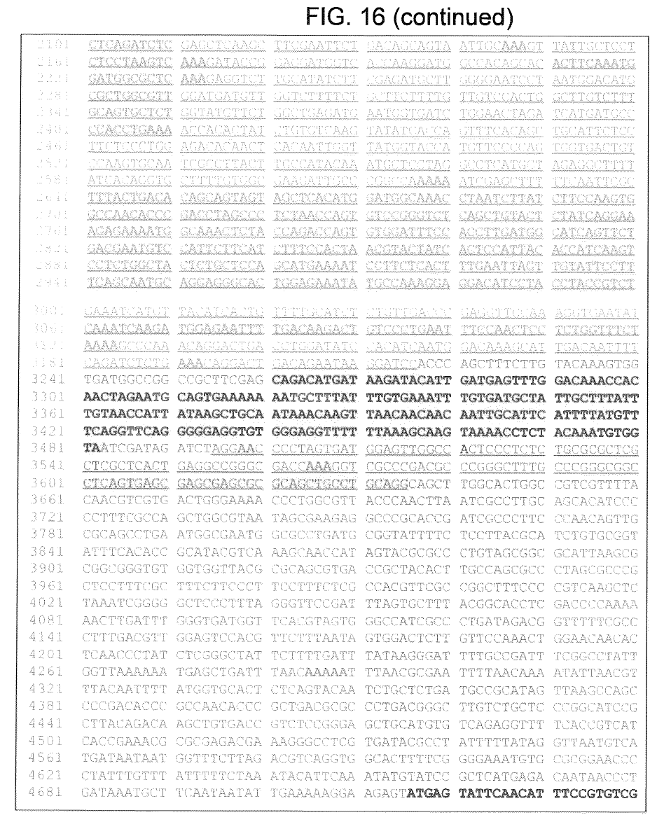

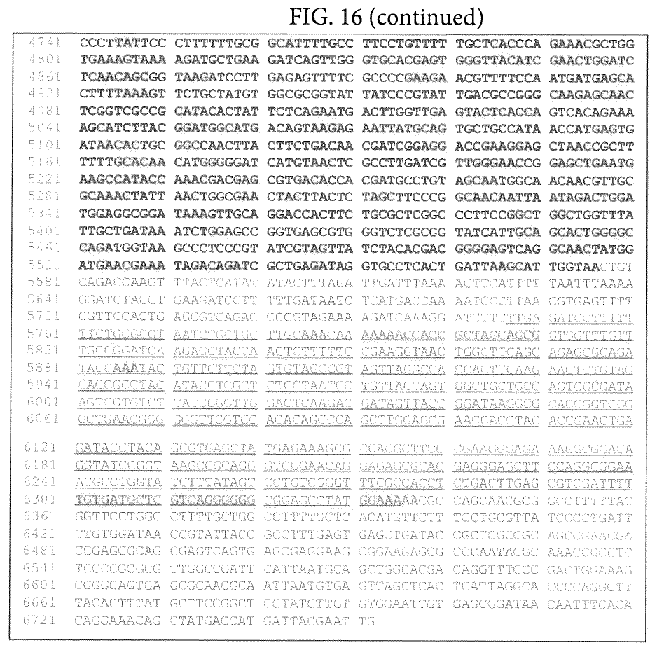

Table 2 and Table 3 in FIG. 16 have the color-coded segments and sequence for the AAV vector encoding Kir7.1 (SEQ ID NO:9).

### AAV Viral Vector Packaging

The adeno-associated virus (AAV) vector system is a popular and versatile tool for in vitro and in vivo gene delivery. AAV is effective in transducing many mammalian cell types, and, unlike adenovirus, has very low immunogenicity, being almost entirely nonpathogenic in vivo. This makes AAV the ideal viral vector system for many animal studies.

An AAV vector is first constructed as a plasmid in E. coli. It is then transfected into packaging cells along with helper plasmids, where the region of the vector between the two inverted terminal repeats (ITRs) is packaged into live virus. When the virus is added to target cells, the double-stranded linear DNA genome is delivered into cells where it enters the nucleus and remains as episomal DNA without integration into the host genome. Any gene(s) placed in-between the two ITRs are introduced into target cells along with the rest of viral genome.

A major practical advantage of AAV is that in most cases AAV can be handled in biosafety level 1 (BSL1) facilities. This is due to AAV being inherently replication-deficient, producing little or no inflammation, and causing no known human disease.

Many strains of AAV have been identified in nature. They are divided into different serotypes based on different antigenicity of the capsid protein on the viral surface. Different serotypes can render the virus with different tissue tropism (i.e. tissue specificity of infection). Different AAV serotypes have tropism for different cell types, and certain cell types may be hard to transduce by any serotype. See, e.g., Curr Opin Pharmacol. 24:59-67 (2015). We found that the AAV2 serotype may be used to effectively transduce retinal pigment epithelium (RPE) cells either in vitro or in vivo. See, e.g., Examples 1 and 2.

### Example 4—Preparation of Lentivirus Viral Vectors for Delivery of Kir7.1 Protein

### Lentivirus Viral Vector Construction

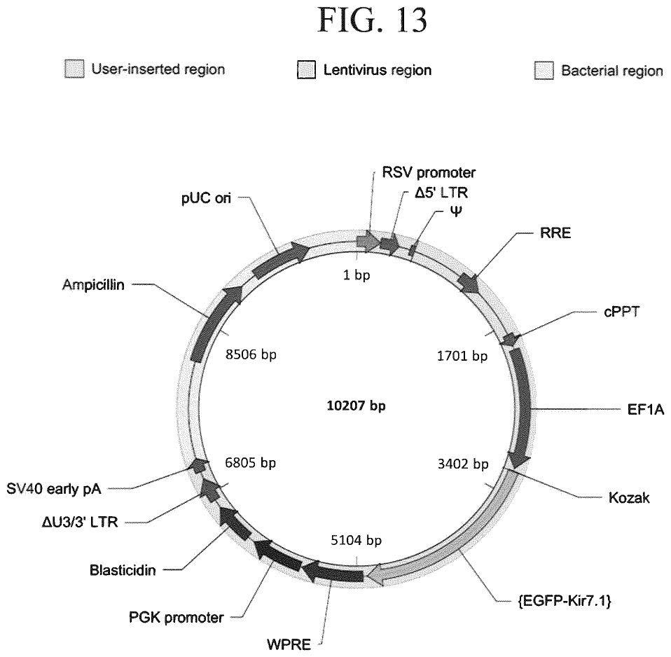

Lentivirus vectors for the delivery of Kir7.1 protein were produced using VectorBuilder software of Cyagen Biosciences and packaging services from Cyagen Biosciences. The following Tables 4-6 and FIG. 13 summarize the construction of Lentivirus vectors that successfully rescued physiological defects in the KCNJ13 gene.

TABLE 4
Vector Summary

| | |
|---|---|
| Vector ID | VB161020-1047mdf |
| Vector Name (official) | pLV[Exp]-Bsd-EF1A>[EGFP-Kir7.1] |
| Date Created (Pacific Time) | 2016 Oct. 19 |
| Size | 10207 bp |
| Vector Type | Lentivirus gene expression vector (3rd generation) |
| Inserted Promoter | EF1A |
| Inserted ORF | [EGFP-Kir7.1] |
| Inserted Marker | Bsd |
| Copy Number | High |
| Bacterial Resistance | Ampicillin |
| Cloning Host | Stbl3 |

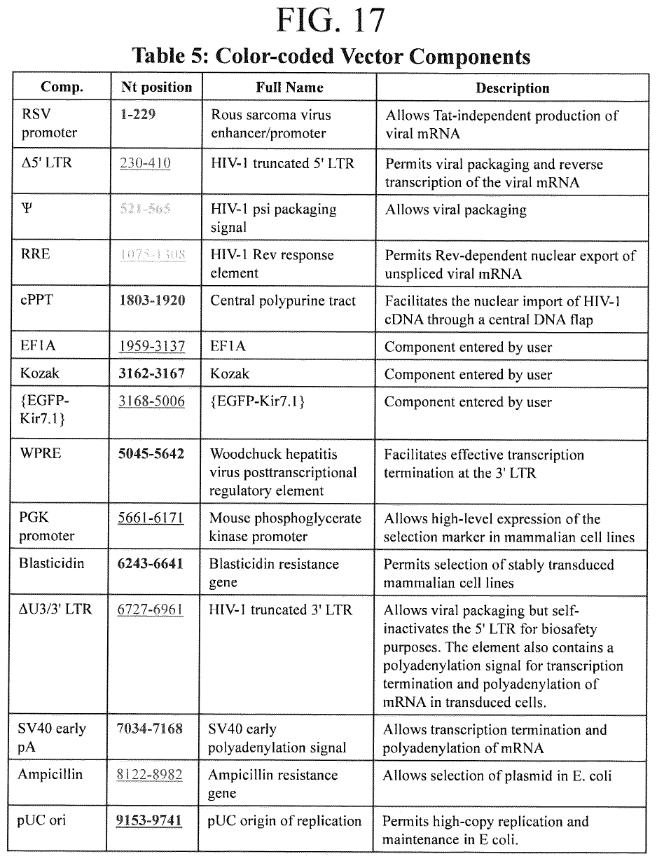

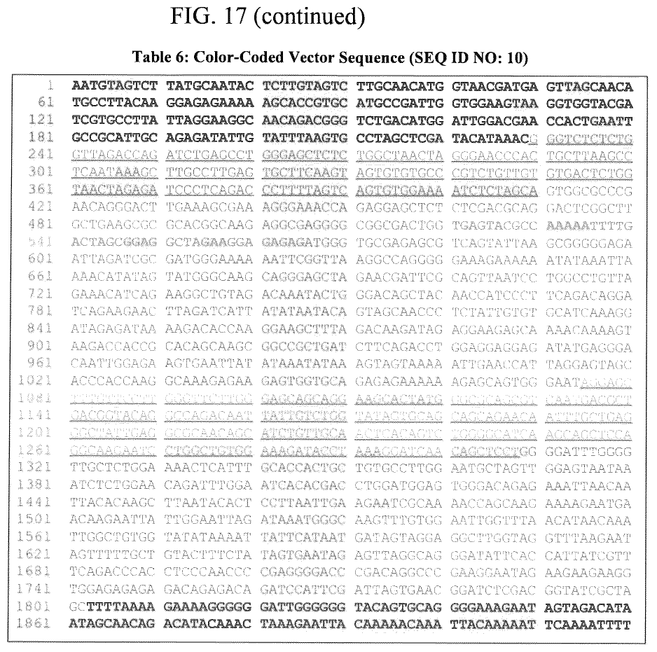

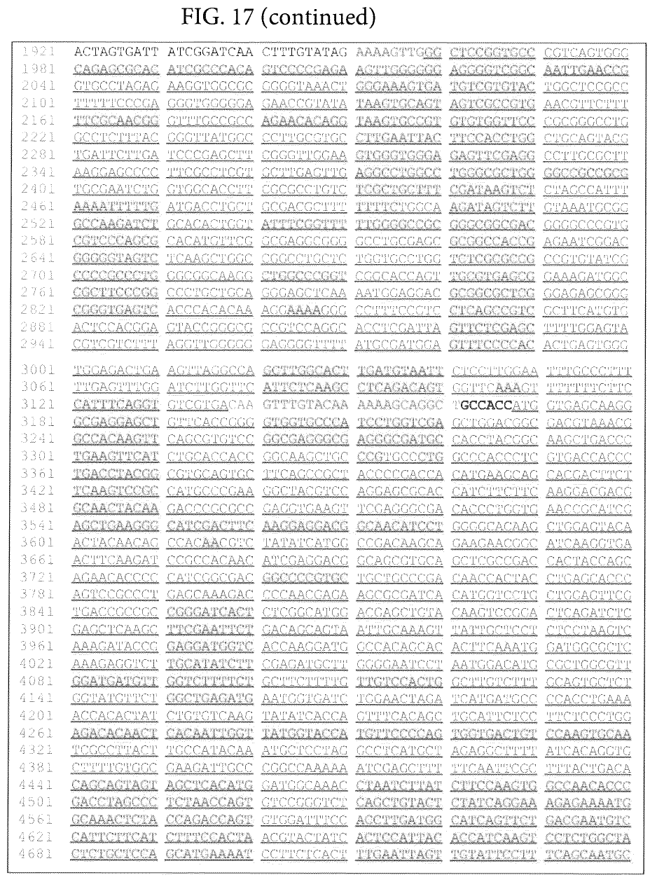

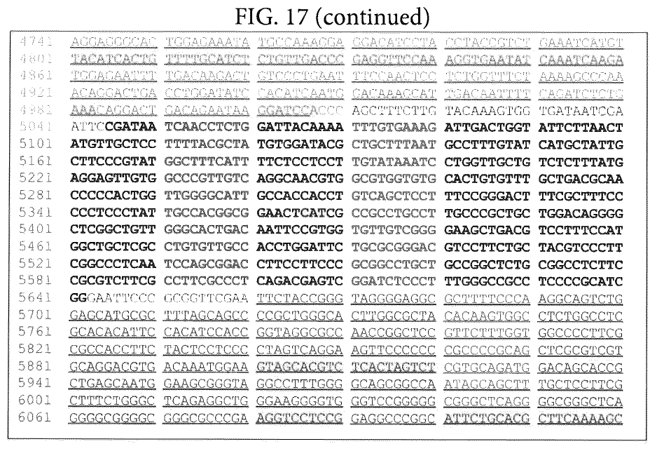

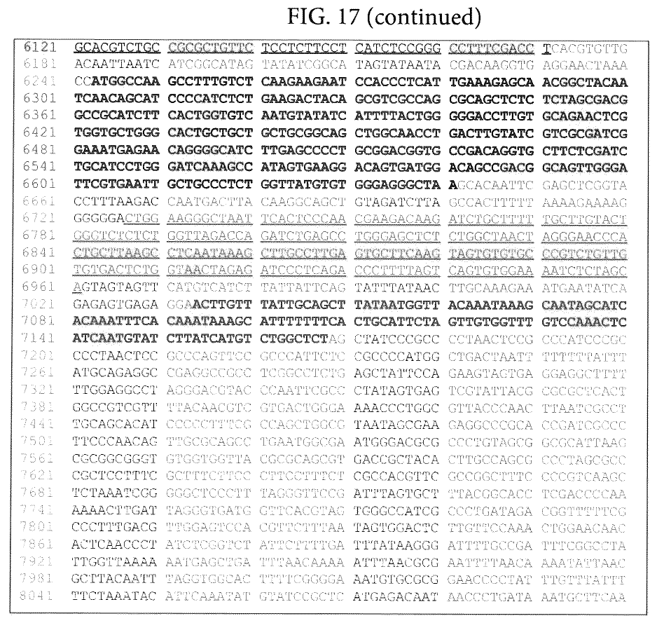

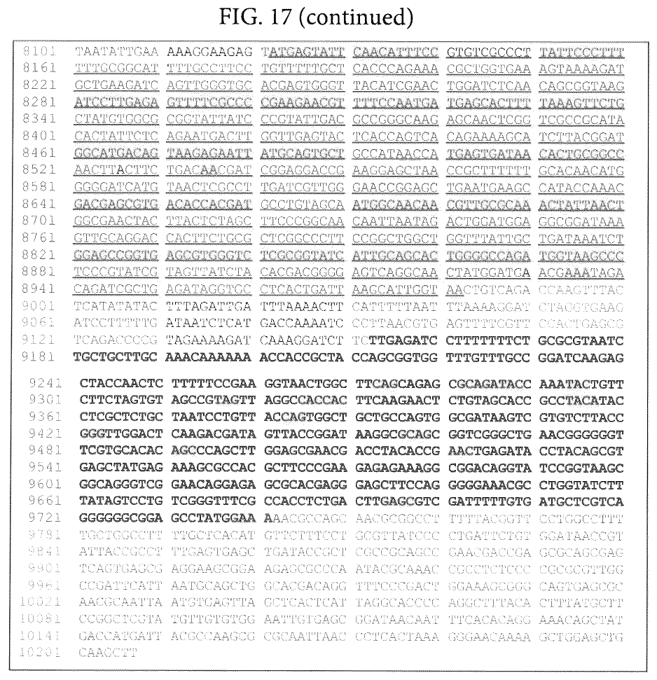

Table 5 and 6 found in FIG. 17 provide the color index and sequence listing for the lentiviral vector (SEQ ID NO:10).

### Lentivirus Viral Vector Packaging

The lentiviral vector system is a highly efficient vehicle for introducing genes permanently into mammalian cells. Lentiviral vectors are derived from HIV, which is a member of the retrovirus family. Wildtype lentivirus has a plus-strand linear RNA genome.

A lentiviral vector is first constructed as a plasmid in E. coli. It is then transfected into packaging cells along with several helper plasmids. Inside the packaging cells, vector DNA located between the two long terminal repeats (LTRs) is transcribed into RNA, and viral proteins expressed by the helper plasmids further package the RNA into virus. Live virus is then released into the supernatant, which can be used to infect target cells directly or after concentration.

By design, lentiviral vectors lack the genes required for viral packaging and transduction (these genes are instead carried by helper plasmids used during virus packaging). As a result, virus produced from lentiviral vectors has the important safety feature of being replication incompetent (meaning that they can transduce target cells but cannot replicate in them).

The Lentivirus viral vectors described herein may be derived from the third-generation lentiviral vector system. See, e.g., J Virol. 72:8463 (1998). It is optimized for high copy number replication in E. coli, high-titer packaging of live virus, efficient viral transduction of a wide range of cells, efficient vector integration into the host genome, and high-level transgene expression.

The packaging system for the lentivirus viral vectors described herein may add the VSV-G envelop protein to the viral surface. This protein has broad tropism and we found that it may help transduce retinal pigment epithelium (RPE) cells either in vitro or in vivo.

We claim:

1. A gene therapy vector comprising a heterologous tissue specific promoter operably connected to a polynucleotide encoding a Kir7.1 polypeptide, wherein the Kir7.1 polypeptide comprises a polypeptide having at least 95% sequence identity to SEQ ID NO: 1, and wherein the Kir7.1. polypeptide is capable of restoring Kir7.1 protein function in retinal pigment epithelial cells lacking such function after delivery of the gene therapy vector to the cells.

2. The gene therapy vector of claim 1, wherein the Kir7.1 polypeptide comprises a polypeptide having at least 98% sequence identity to SEQ ID NO: 1.

3. The gene therapy vector of claim 1, wherein the heterologous tissue specific promoter is active in the retinal pigment epithelium (RPE) in the eye of a subject.

4. The gene therapy vector of claim 1, wherein the heterologous tissue specific promoter is an EF1a promoter comprising at least 95% sequence identity to SEQ ID NO: 3.

5. The gene therapy vector of claim 1, wherein the heterologous tissue specific promoter is a VMD2 promoter comprising at least 95% sequence identity to SEQ ID NO: 4.

6. The gene therapy vector of claim 1, wherein the gene therapy vector is a viral vector.

7. The gene therapy vector of claim 6, wherein the viral vector is selected from the group consisting of a retroviral vector, an adeno-associated viral (AAV) vector, and an adenoviral vector.

8. The gene therapy vector of claim 7, wherein the viral vector is a lentiviral vector.

9. The gene therapy vector of claim 7, wherein the viral vector is an adeno-associated viral vector (AAV).

10. The gene therapy vector of claim 9, wherein the AAV vector is an AAV2 vector.

11. The gene therapy vector of claim 6, wherein the viral vector is a virus particle and comprises a VSV-G envelope protein.

12. A lentiviral vector or adeno-associated viral (AAV) vector comprising a polynucleotide having at least 95% sequence identity to SEQ ID NO: 5 (EF1a-Kir7.1) or SEQ ID NO: 6 (VMD2-Kir7.1) and wherein the vector is capable of restoring Kir7.1 protein function in retinal pigment epithelial cells lacking such function after delivery of the vector to the cells.

13. A therapeutic composition comprising the gene therapy vectors of claim 1 and a pharmaceutically-acceptable carrier.

14. A method of treating a subject having a condition associated with insufficient expression or function of a Kir7.1 polypeptide comprising administering a therapeutically effective amount of a gene therapy vector comprising a heterologous tissue specific promoter operably connected to a polynucleotide encoding a Kir7.1 polypeptide, wherein the Kir7.1 polypeptide comprises a polypeptide having at least 95% sequence identity to SEQ ID NO: 1.

15. The method of claim 14, wherein the condition is associated with at least one loss-of-function mutation in a KCNJ13 gene.

16. The method of claim 15, wherein the at least one loss-of-function mutation results in a substitution to SEQ ID NO:1 selected from the group consisting of W53Ter, Q116R, I120T, T153I, R162Q, R166Ter, L241P, E276A, S105I, and G219Ter.

17. The method of claim 14, wherein the condition is selected from the group consisting of Leber Congenital Amaurosis 16 (LCA16), retinitis pigmentosa, and Snowflake Vitreoretinal Degeneration (SVD).

18. The method of claim 14, wherein the gene therapy vector is administered subretinally to at least one eye of the subject.

19. The method of claim 14, wherein between 10⁶ and 10¹⁴ copies of the gene therapy vector are administered to the subject.

20. A method of expressing a Kir7.1 polypeptide in a retinal pigment epithelium (RPE) cell lacking a functional Kir7.1 polypeptide comprising contacting the RPE cell with an adeno-associated viral 2 (AAV2) viral particle comprising a promoter operably connected to the Kir7.1 polypeptide, wherein expression of the Kir7.1 polypeptide in the RPE cells restores the electrophysiological phenotype of the RPE cell lacking the functional Kir7.1 polypeptide.


SEQUENCE LISTING (20 sequences)

SEQ ID NO: 1 - protein, 360 aa, Homo sapiens
MDSSNCKVIAPLLSQRYRRMVTKDGHSTLQMDGAQRGLAYLRDAWGILMDMRWRWMMLVFSASFVVHWLVFAVLWYVLAEMNGDLELDHDAPPENHTICVKYITSFTAAFSFSLETQLTIGYGTMFPSGDCPSAIALLAIQMLLGLMLEAFITGAFVAKIARPKNRAFSIRFTDTAVVAHMDGKPNLIFQVANTRPSPLTSVRVSAVLYQERENGKLYQTSVDFHLDGISSDECPFFIFPLTYYHSITPSSPLATLLQHENPSHFELVVFLSAMQEGTGEICQRRTSYLPSEIMLHHCFASLLTRGSKGEYQIKMENFDKTVPEFPTPLVSKSPNRTDLDIHINGQSIDNFQISETGLTE

SEQ ID NO: 2 - DNA, 1083 bp, Homo sapiens
  source 1..1083: mol_type = other DNA
atggacagcagtaattgcaaagttattgctcctctcctaagtcaaagataccggaggatggtcaccaaggatggccacagcacacttcaaatggatggcgctcaaagaggtcttgcatatcttcgagatgcttggggaatcctaatggacatgcgctggcgttggatgatgttggtcttttctgcttcttttgttgtccactggcttgtctttgcagtgctctggtatgttctggctgagatgaatggtgatctggaactagatcatgatgccccacctgaaaaccacactatctgtgtcaagtatatcaccagtttcacagctgcattctccttctccctggagacacaactcacaattggttatggtaccatgttccccagtggtgactgtccaagtgcaatcgccttacttgccatacaaatgctcctaggcctcatgctagaggcttttatcacaggtgcttttgtggcgaagattgcccggccaaaaaatcgagctttttcaattcgctttactgacatagcagtagtagctcacatggatggcaaacctaatcttatcttccaagtggccaacacccgacctagccctctaaccagtgtccgggtctcagctgtactctatcaggaaagagaaaatggcaaactctaccagaccagtgtggatttccaccttgatggcatcagttctgacgaatgtccattcttcatctttccactaacgtactatcactccattacaccatcaagtcctctggctactctgctccagcatgaaaatccttctcactttgaattagttgtattcctttcagcaatgcaggagggcactggagaaatatgccaaaggaggacatcctacctacagtctgaaatcatgttacatcactgttttgcatctctgttgacccgaggttccaaatgtgaatatcaaatcaagatggagaattttgacaagactgtccctgaatttccaactcctctggtttctaaaagcccaaacaggactgacctggatatccacatcaatggacaaagcattgacaattttcagatctctgaaacaggactgacagaataa

SEQ ID NO: 3 - DNA, 1179 bp, Homo sapiens
  source 1..1179: mol_type = other DNA
ggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggtctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtga

SEQ ID NO: 4 - DNA, 626 bp, Homo sapiens
  source 1..626: mol_type = other DNA
aattctgtcattttactagggtgatgaaattcccaagcaacaccatccttttcagataagggcactgaggctgagagaggagctgaaacctacccggggtcaccacacacaggtggcaaggctgggaccagaaaccaggactgttgactgcagcccggtattcattctttccatagcccacagggctgtcaaagaccccagggcctagtcagaggctcctccttcctggagagttcctggcacagaagttgaagctcagcacagccccctaacccccaactctctctgcaaggcctcaggggtcagaacactggtggagcagatcctttagcctctggattttagggccatggtagagggggtgttgccctaaattccagccctggtctcagcccaacaccctccaagaagaaattagaggggccatggccaggctgtgctagccgttgcttctgagcagattacaagaagggactaagacaaggactcctttgtggaggtcctggcttagggagtcaagtgacggcggctcagcactcacgtgggcagtgccagcctctaagagtgggcaggggcactggccacagagtcccagggagtcccaccagcctagtcgccaggtcgac

SEQ ID NO: 5 - DNA, 3048 bp, synthetic construct
  misc_feature 1..3048: note = synthetic
  source 1..3048: mol_type = other DNA
ggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggtctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgacaagtttgtacaaaaaagcaggctgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtccggactcagatctcgagctcaagcttcgaattctgacagcagtaattgcaaagttattgctcctctcctaagtcaaagataccggaggatggtcaccaaggatggccacagcacacttcaaatggatggcgctcaaagaggtcttgcatatcttcgagatgcttggggaatcctaatggacatgcgctggcgttggatgatgttggtcttttctgcttcttttgttgtccactggcttgtctttgcagtgctctggtatgttctggctgagatgaatggtgatctggaactagatcatgatgccccacctgaaaaccacactatctgtgtcaagtatatcaccagtttcacagctgcattctccttctccctggagacacaactcacaattggttatggtaccatgttccccagtggtgactgtccaagtgcaatcgccttacttgccatacaaatgctcctaggcctcatgctagaggcttttatcacaggtgcttttgtggcgaagattgcccggccaaaaaatcgagctttttcaattcgctttactgacacagcagtagtagctcacatggatggcaaacctaatcttatcttccaagtggccaacacccgacctagccctctaaccagtgtccgggtctcagctgtactctatcaggaaagagaaaatggcaaactctaccagaccagtgtggatttccaccttgatggcatcagttctgacgaatgtccattcttcatctttccactaacgtactatcactccattacaccatcaagtcctctggctactctgctccagcatgaaaatccttctcactttgaattagttgtattcctttcagcaatgcaggagggcactggagaaatatgccaaaggaggacatcctacctaccgtctgaaatcatgttacatcactgttttgcatctctgttgacccgaggttccaaaggtgaatatcaaatcaagatggagaattttgacaagactgtccctgaatttccaactcctctggtttctaaaagcccaaacaggactgacctggatatccacatcaatggacaaagcattgacaattttcagatctctgaaacaggactgacagaataaggatcc

SEQ ID NO: 6 - DNA, 2495 bp, synthetic construct
  misc_feature 1..2495: note = synthetic
  source 1..2495: mol_type = other DNA
aattctgtcattttactagggtgatgaaattcccaagcaacaccatccttttcagataagggcactgaggctgagagaggagctgaaacctacccggggtcaccacacacaggtggcaaggctgggaccagaaaccaggactgttgactgcagcccggtattcattctttccatagcccacagggctgtcaaagaccccagggcctagtcagaggctcctccttcctggagagttcctggcacagaagttgaagctcagcacagccccctaacccccaactctctctgcaaggcctcaggggtcagaacactggtggagcagatcctttagcctctggattttagggccatggtagagggggtgttgccctaaattccagccctggtctcagcccaacaccctccaagaagaaattagaggggccatggccaggctgtgctagccgttgcttctgagcagattacaagaagggactaagacaaggactcctttgtggaggtcctggcttagggagtcaagtgacggcggctcagcactcacgtgggcagtgccagcctctaagagtgggcaggggcactggccacagagtcccagggagtcccaccagcctagtcgccaggtcgaccaagtttgtacaaaaaagcaggctgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtccggactcagatctcgagctcaagcttcgaattctgacagcagtaattgcaaagttattgctcctctcctaagtcaaagataccggaggatggtcaccaaggatggccacagcacacttcaaatggatggcgctcaaagaggtcttgcatatcttcgagatgcttggggaatcctaatggacatgcgctggcgttggatgatgttggtcttttctgcttcttttgttgtccactggcttgtctttgcagtgctctggtatgttctggctgagatgaatggtgatctggaactagatcatgatgccccacctgaaaaccacactatctgtgtcaagtatatcaccagtttcacagctgcattctccttctccctggagacacaactcacaattggttatggtaccatgttccccagtggtgactgtccaagtgcaatcgccttacttgccatacaaatgctcctaggcctcatgctagaggcttttatcacaggtgcttttgtggcgaagattgcccggccaaaaaatcgagctttttcaattcgctttactgacacagcagtagtagctcacatggatggcaaacctaatcttatcttccaagtggccaacacccgacctagccctctaaccagtgtccgggtctcagctgtactctatcaggaaagagaaaatggcaaactctaccagaccagtgtggatttccaccttgatggcatcagttctgacgaatgtccattcttcatctttccactaacgtactatcactccattacaccatcaagtcctctggctactctgctccagcatgaaaatccttctcactttgaattagttgtattcctttcagcaatgcaggagggcactggagaaatatgccaaaggaggacatcctacctaccgtctgaaatcatgttacatcactgttttgcatctctgttgacccgaggttccaaaggtgaatatcaaatcaagatggagaattttgacaagactgtccctgaatttccaactcctctggtttctaaaagcccaaacaggactgacctggatatccacatcaatggacaaagcattgacaattttcagatctctgaaacaggactgacagaataaggatcc

SEQ ID NO: 7 - DNA, 26 bp, synthetic construct
  misc_feature 1..26: note = synthetic
  source 1..26: mol_type = other DNA
gcttcgaattccgacagcagtaattg

SEQ ID NO: 8 - DNA, 26 bp, synthetic construct
  misc_feature 1..26: note = synthetic
  source 1..26: mol_type = other DNA
atccggtggatccttattctgtcagt

SEQ ID NO: 9 - DNA, 6752 bp, synthetic construct
  misc_feature 1..6752: note = synthetic
  source 1..6752: mol_type = other DNA
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctatcgatcaactttgtatagaaaagttgggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggtctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgacaagtttgtacaaaaaagcaggctgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtccggactcagatctcgagctcaagcttcgaattctgacagcagtaattgcaaagttattgctcctctcctaagtcaaagataccggaggatggtcaccaaggatggccacagcacacttcaaatggatggcgctcaaagaggtcttgcatatcttcgagatgcttggggaatcctaatggacatgcgctggcgttggatgatgttggtcttttctgcttcttttgttgtccactggcttgtctttgcagtgctctggtatgttctggctgagatgaatggtgatctggaactagatcatgatgccccacctgaaaaccacactatctgtgtcaagtatatcaccagtttcacagctgcattctccttctccctggagacacaactcacaattggttatggtaccatgttccccagtggtgactgtccaagtgcaatcgccttacttgccatacaaatgctcctaggcctcatgctagaggcttttatcacaggtgcttttgtggcgaagattgcccggccaaaaaatcgagctttttcaattcgctttactgacacagcagtagtagctcacatggatggcaaacctaatcttatcttccaagtggccaacacccgacctagccctctaaccagtgtccgggtctcagctgtactctatcaggaaagagaaaatggcaaactctaccagaccagtgtggatttccaccttgatggcatcagttctgacgaatgtccattcttcatctttccactaacgtactatcactccattacaccatcaagtcctctggctactctgctccagcatgaaaatccttctcactttgaattagttgtattcctttcagcaatgcaggagggcactggagaaatatgccaaaggaggacatcctacctaccgtctgaaatcatgttacatcactgttttgcatctctgttgacccgaggttccaaaggtgaatatcaaatcaagatggagaattttgacaagactgtccctgaatttccaactcctctggtttctaaaagcccaaacaggactgacctggatatccacatcaatggacaaagcattgacaattttcagatctctgaaacaggactgacagaataaggatccacccagctttcttgtacaaagtggtgatggccggccgcttcgagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtaatcgatagatctaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggcagcttggcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaaccctatctcgggctattcttttgatttataagggattttgccgatttcggcctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgaattg

SEQ ID NO: 10 - DNA, 10207 bp, synthetic construct
  misc_feature 1..10207: note = synthetic
  source 1..10207: mol_type = other DNA
aatgtagtcttatgcaatactcttgtagtcttgcaacatggtaacgatgagttagcaacatgccttacaaggagagaaaaagcaccgtgcatgccgattggtggaagtaaggtggtacgatcgtgccttattaggaaggcaacagacgggtctgacatggattggacgaaccactgaattgccgcattgcagagatattgtatttaagtgcctagctcgatacataaacgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggtatcgctagcttttaaaagaaaaggggggattggggggtacagtgcaggggaaagaatagtagacataatagcaacagacatacaaactaaagaattacaaaaacaaattacaaaaattcaaaattttactagtgattatcggatcaactttgtatagaaaagttgggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggtctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgacaagtttgtacaaaaaagcaggctgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtccggactcagatctcgagctcaagcttcgaattctgacagcagtaattgcaaagttattgctcctctcctaagtcaaagataccggaggatggtcaccaaggatggccacagcacacttcaaatggatggcgctcaaagaggtcttgcatatcttcgagatgcttggggaatcctaatggacatgcgctggcgttggatgatgttggtcttttctgcttcttttgttgtccactggcttgtctttgcagtgctctggtatgttctggctgagatgaatggtgatctggaactagatcatgatgccccacctgaaaaccacactatctgtgtcaagtatatcaccagtttcacagctgcattctccttctccctggagacacaactcacaattggttatggtaccatgttccccagtggtgactgtccaagtgcaatcgccttacttgccatacaaatgctcctaggcctcatgctagaggcttttatcacaggtgcttttgtggcgaagattgcccggccaaaaaatcgagctttttcaattcgctttactgacacagcagtagtagctcacatggatggcaaacctaatcttatcttccaagtggccaacacccgacctagccctctaaccagtgtccgggtctcagctgtactctatcaggaaagagaaaatggcaaactctaccagaccagtgtggatttccaccttgatggcatcagttctgacgaatgtccattcttcatctttccactaacgtactatcactccattacaccatcaagtcctctggctactctgctccagcatgaaaatccttctcactttgaattagttgtattcctttcagcaatgcaggagggcactggagaaatatgccaaaggaggacatcctacctaccgtctgaaatcatgttacatcactgttttgcatctctgttgacccgaggttccaaaggtgaatatcaaatcaagatggagaattttgacaagactgtccctgaatttccaactcctctggtttctaaaagcccaaacaggactgacctggatatccacatcaatggacaaagcattgacaattttcagatctctgaaacaggactgacagaataaggatccacccagctttcttgtacaaagtggtgataatcgaattccgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgggaattcccgcggttcgaattctaccgggtaggggaggcgcttttcccaaggcagtctggagcatgcgctttagcagccccgctgggcacttggcgctacacaagtggcctctggcctcgcacacattccacatccaccggtaggcgccaaccggctccgttctttggtggccccttcgcgccaccttctactcctcccctagtcaggaagttcccccccgccccgcagctcgcgtcgtgcaggacgtgacaaatggaagtagcacgtctcactagtctcgtgcagatggacagcaccgctgagcaatggaagcgggtaggcctttggggcagcggccaatagcagctttgctccttcgctttctgggctcagaggctgggaaggggtgggtccgggggcgggctcaggggcgggctcaggggcggggcgggcgcccgaaggtcctccggaggcccggcattctgcacgcttcaaaagcgcacgtctgccgcgctgttctcctcttcctcatctccgggcctttcgacctcacgtgttgacaattaatcatcggcatagtatatcggcatagtataatacgacaaggtgaggaactaaaccatggccaagcctttgtctcaagaagaatccaccctcattgaaagagcaacggctacaatcaacagcatccccatctctgaagactacagcgtcgccagcgcagctctctctagcgacggccgcatcttcactggtgtcaatgtatatcattttactgggggaccttgtgcagaactcgtggtgctgggcactgctgctgctgcggcagctggcaacctgacttgtatcgtcgcgatcggaaatgagaacaggggcatcttgagcccctgcggacggtgccgacaggtgcttctcgatctgcatcctgggatcaaagccatagtgaaggacagtgatggacagccgacggcagttgggattcgtgaattgctgccctctggttatgtgtgggagggctaagcacaattcgagctcggtacctttaagaccaatgacttacaaggcagctgtagatcttagccactttttaaaagaaaaggggggactggaagggctaattcactcccaacgaagacaagatctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtagtagttcatgtcatcttattattcagtatttataacttgcaaagaaatgaatatcagagagtgagaggaacttgtttattgcagcttataatggttacaaataaagcaatagcatcacaaatttcacaaataaagcatttttttcactgcattctagttgtggtttgtccaaactcatcaatgtatcttatcatgtctggctctagctatcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctagggacgtacccaattcgccctatagtgagtcgtattacgcgcgctcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagagagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcgcgcaattaaccctcactaaagggaacaaaagctggagctgcaagctt

SEQ ID NO: 11 - DNA, 31 bp, synthetic construct
  misc_feature 1..31: note = synthetic
  source 1..31: mol_type = other DNA
ccgctcgagtaccttccaagtgctgtcaaac

SEQ ID NO: 12 - DNA, 30 bp, synthetic construct
  misc_feature 1..30: note = synthetic
  source 1..30: mol_type = other DNA
cgacgcgtcatgctgaattccttaatttgc

SEQ ID NO: 13 - DNA, 30 bp, synthetic construct
  misc_feature 1..30: note = synthetic
  source 1..30: mol_type = other DNA
ctagctagctcctcccagcgtaacgtgagc

SEQ ID NO: 14 - DNA, 28 bp, synthetic construct
  misc_feature 1..28: note = synthetic
  source 1..28: mol_type = other DNA
gaagatctctagtggcagccccatggtg

SEQ ID NO: 15 - DNA, 32 bp, synthetic construct
  misc_feature 1..32: note = synthetic
  source 1..32: mol_type = other DNA
ctagctagcctgtcctcttaggcagatacaga

SEQ ID NO: 16 - DNA, 30 bp, synthetic construct
  misc_feature 1..30: note = synthetic
  source 1..30: mol_type = other DNA
gaagatctagagccttcatgttgactgcta

SEQ ID NO: 17 - DNA, 20 bp, synthetic construct
  misc_feature 1..20: note = synthetic
  source 1..20: mol_type = other DNA
gaaagttggggatgaggcga

SEQ ID NO: 18 - DNA, 33 bp, synthetic construct
  misc_feature 1..33: note = synthetic
  source 1..33: mol_type = other DNA
caatcaaagcttcctcagagctgccgggcggct

SEQ ID NO: 19 - DNA, 21 bp, synthetic construct
  misc_feature 1..21: note = synthetic
  source 1..21: mol_type = other DNA
gacatgcgctggcgttggatg

SEQ ID NO: 20 - DNA, 21 bp, synthetic construct
  misc_feature 1..21: note = synthetic
  source 1..21: mol_type = other DNA
gacatgcgctagcgttggatg